(19) United States
(12) Patent Application Publication
(10) Pub. No.: US 2021/0085229 A1
(43) Pub. Date: Mar. 25, 2021

(54) BIOMETRIC MEASUREMENT APPARATUS AND BIOMETRIC MEASUREMENT METHOD

(71) Applicant: Panasonic Intellectual Property Management Co., Ltd., Osaka (JP)

(72) Inventors: KENJI NARUMI, Osaka (JP); TSUGUHIRO KORENAGA, Osaka (JP)

(21) Appl. No.: 16/953,217

(22) Filed: Nov. 19, 2020

Related U.S. Application Data
(63) Continuation of application No. PCT/JP2019/028605, filed on Jul. 22, 2019.

(30) Foreign Application Priority Data
Aug. 30, 2018 (JP) 2018-162142

(51) Int. Cl. A61B 5/1455; A61B 5/026; A61B 5/00

(52) U.S. Cl. CPC A61B 5/14553; A61B 5/0261; A61B 5/4058; A61B 5/6893; A61B 5/14552

(57) ABSTRACT

A biometric measurement apparatus includes a light source that emits light onto a head portion of a user, an image sensor, a controller that controls the light source and the image sensor, and a signal processor. The controller causes the light source to emit the light and causes the image sensor to output an image signal by causing the image sensor to detect at least part of reflected light returning from the head portion in response to emission of the light. The signal processor generates brain activity data indicating a state of a brain of the user and stops outputting the brain activity data based on at least one selected from the group consisting of the image signal and a sensor signal output from a sensor that detects a change, affecting the brain activity data, in an environment surrounding the user.

### BACKGROUND

### 1. Technical Field

The present disclosure relates to a biometric measurement apparatus and a biometric measurement method.

### 2. Description of the Related Art

In a technique available in the field of biometric measurement, light is emitted to a target object and internal information on the target object is acquired from light transmitted through the target object. In this technique, a surface reflection component reflected from the surface of the target object may sometimes become noise. If the noise due to the surface reflection component is removed, desired internal information may be more accurately acquired.

Japanese Unexamined Patent Application Publication No. 2017-202328 discloses an imaging apparatus that acquires, in a non-contact fashion, internal information on a target object in a state with the noise due to the surface reflection component reduced.

### SUMMARY

In one general aspect, the techniques disclosed here feature a biometric measurement apparatus. The biometric measurement apparatus includes a light source that emits light onto a head portion of a user, an image sensor, a controller that controls the light source and the image sensor, and a signal processor. The controller causes the light source to emit the light and causes the image sensor to output an image signal by causing the image sensor to detect at least part of reflected light returning from the head portion in response to emission of the light. The signal processor generates brain activity data indicating a state of a brain of the user based on the image signal and stops outputting the brain activity data based on at least one selected from the group consisting of the image signal and a sensor signal output from a sensor that detects a change in an environment surrounding the user, the change affecting the brain activity data.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A schematically illustrates an example of a biometric measurement apparatus of an embodiment;

FIG. 1B schematically illustrates a pixel in an image sensor;

FIG. 1C illustrates a structure example of the image sensor;

FIG. 1D schematically illustrates an operation within a frame according to the embodiment;

FIG. 1E is a flowchart illustrating a process of a light source and image sensor controlled by a control circuit;

FIG. 1F schematically illustrates an optical signal that arrives at the image sensor when a rectangular light pulse emitted from a light source returns from a user;

FIG. 1G schematically illustrates an example of a timing chart in detecting a surface reflection component;

FIG. 1H schematically illustrates an example of a timing chart in detecting an internal scatter component;

FIG. 2 is a flowchart illustrating a process example that measures biometric measurement data on a user in accordance with the embodiment;

FIG. 3A illustrates a process example that measures the biometric measurement data on the user in accordance with the embodiment;

FIG. 3B schematically illustrates a relationship between a change in body motion and an invalid time period;

FIG. 3C schematically illustrates a relationship between a motion speed of a head and an invalid time period;

FIG. 3D illustrates a process example that measures the biometric measurement data on the user in accordance with the embodiment;

FIG. 3E schematically illustrates an example of a biometric measurement apparatus of the embodiment;

FIG. 4 schematically illustrates an example of a biometric measurement apparatus of the embodiment;

FIG. 5A is a flowchart illustrating a process example that measures biometric measurement data on the user in accordance with the embodiment;

FIG. 5B illustrates a relationship between a difference value and reliability;

FIG. 6 illustrates a process example that measures the biometric measurement data on the user in accordance with the embodiment;

FIG. 7A schematically illustrates an example of a biometric measurement apparatus of the embodiment;

FIG. 7B schematically illustrates an installation example of each element of the biometric measurement apparatus in an automobile;

FIG. 7C schematically illustrates an installation example of each element of the biometric measurement apparatus that is mounted on a game machine or an attraction device;

FIG. 8 is a flowchart illustrating a process example that measures biometric measurement data on the user in accordance with the embodiment;

FIG. 9 illustrates a process example that measures biometric measurement data on the user in accordance with the embodiment;

FIG. 10A illustrates a relationship between the distance of motion of the head and time; and

FIG. 10B illustrates a relationship between oxidized hemoglobin concentration and time.

### DETAILED DESCRIPTION

### Underlying Knowledge Forming Basis of the Present Disclosure

It is known in the field of biometric measurement that a change in the state of a user affects measurement results. For example, in the measurement of brain activity using near infrared spectroscopy (NIRS), the motion of the head of the user is referred to as body motion. The body motion is believed to cause irregular variations in a signal representing a change in cerebral blood flow.

If the measurement of brain activity is performed for research applications, an operator of NIRS device may determine irregularity in the signal, notify a user that the measurement is in error, and prompt the user to perform the measurement again. However, such a sequence is difficult to take in an application where the user measure brain activity on a daily basis.

If the measurement apparatus measures brain activity from the head of the user in a non-contact fashion, the motion of the head varies a relative positional relationship between the measurement apparatus and the user. This even further affects a detection signal related to a change in the cerebral flood flow.

Japanese Unexamined Patent Application Publication No. 2017-202328 discloses a method of correcting a position displacement responsive to a motion by pattern-matching cerebral blood flow distributions between frames. The pattern of the cerebral blood flow distribution has a low spatial frequency. Specifically, the cerebral blood flow distribution is spatially gentle. For this reason, detecting a feature point for matching is not so easy. Restoring the cerebral blood flow distribution in a manner free from any motion of the head from frame to frame involves a higher computational cost.

The inventors have studied as described above and come up with a biometric measurement apparatus in terms of the items listed as below.

### Item 1

A biometric measurement apparatus according to item 1 includes a light source that emits light onto a head portion of a user, an image sensor, a controller that controls the light source and the image sensor, and a signal processor. The controller causes the light source to emit the light and causes the image sensor to output an image signal by causing the image sensor to detect at least part of reflected light returning from the head portion in response to emission of the light. The signal processor generates brain activity data indicating a state of a brain of the user based on the image signal and stops outputting the brain activity data based on at least one selected from the group consisting of the image signal and a sensor signal output from a sensor that detects a change in an environment surrounding the user, the change affecting the brain activity data.

In the biometric measurement apparatus according to another aspect of the disclosure, if a target object moves during measurement or the environment surrounding the target object changes, internal information on the target object may be measured by using a low-cost method.

### Item 2

In the biometric measurement apparatus according to item 1, the light may be a light pulse, and the controller may cause the image sensor to output as the image signal a first signal that is obtained by detecting a component included in a reflected light pulse during a time period from start to end of a decrease of an intensity of the reflected light pulse. The reflected light pulse returns from the head portion in response to emission of the light pulse.

### Item 3

In the biometric measurement apparatus according to one of items 1 and 2, the controller may cause the light source to repeatedly emit the light during a specific time period and cause the image sensor to repeatedly output the image signal during the specific time period. The signal processor may calculate a first value based on at least one selected from the group consisting of the image signal and the sensor signal and stop outputting the brain activity data during a first time period of the specific time period. The first value satisfies a preset condition during the first time period.

### Item 4

In the biometric measurement apparatus according to item 3, the signal processor may output a signal indicating that the brain activity data remains invalid during the first time period.

### Item 5

In the biometric measurement apparatus according to item 3, the signal processor may output, during the first time period, data identical to the brain activity data that is generated prior to the first time period.

### Item 6

In the biometric measurement apparatus according to item 3, the signal processor may output, as the brain activity data during the first time period, data that is obtained by interpolating between the brain activity data generated prior to the first time period and the brain activity data generated subsequent to the first time period.

### Item 7

In the biometric measurement apparatus according to item 3, the signal processor may further stop outputting the brain activity data during at least a time period selected from the group consisting of a second time period prior to a start of the first time period and a third time period subsequent to an end of the second time period.

### Item 8

In the biometric measurement apparatus according to item 7, the third time period may be longer in time length than the second time period.

### Item 9

In the biometric measurement apparatus according to one of items 3 through 8, a frequency of calculating the first value may be higher than or equal to a frequency of generating the brain activity data.

### Item 10

In the biometric measurement apparatus according to one of items 1 through 9, the light may be a light pulse, and the controller may cause the image sensor to output as the image signal a second signal that is obtained by detecting, a component included in a reflected light pulse prior to a start of a decrease of an intensity of the reflected light pulse returning from the head portion in response to emission of the light pulse. The signal processor may stop outputting the brain activity data based on the second signal.

### Item 11

In the biometric measurement apparatus according to item 10, the signal processor may calculate, based on the second signal, a displacement from a reference position of the head portion or a motion speed of the head portion and stop outputting the brain activity data when an absolute value of the displacement or an absolute value of the motion speed exceeds a threshold.

### Item 12

In the biometric measurement apparatus according to item 10, the signal processor may calculate, based on the second signal, a luminance of the head portion or a change rate in the luminance of the head portion and stop outputting the brain activity data when an absolute value of the luminance or an absolute value of the change rate in the luminance exceeds a threshold.

### Item 13

In the biometric measurement apparatus according to item 10, the signal processor may calculate an area of a specific region in the head portion based on the second signal and stop outputting the brain activity data if the area is smaller than a threshold.

### Item 14

In the biometric measurement apparatus according to one of items 1 through 9, the signal processor may calculate a second value by using the brain activity data and stop outputting the brain activity data when an absolute value of a change rate in the second value exceeds a threshold.

### Item 15

In the biometric measurement apparatus according to item 1, the sensor may be an acceleration sensor installed in the environment surrounding the user.

### Item 16

In the biometric measurement apparatus according to item 1, the sensor may be an illuminance sensor installed in the environment surrounding the user.

### Item 17

In the biometric measurement apparatus according to item 1, the sensor may include at least one selected from the group consisting of a steering angle sensor, a gear position sensor, and a speed sensor, installed in a vehicle to be driven by the user.

### Item 18

A biometric measurement apparatus according to item 18 includes a light source that emits light onto a head portion of a user, an image sensor, a controller that controls the light source and the image sensor, and a signal processor. The controller causes the light source to emit the light and causes the image sensor to output an image signal by causing the image sensor to detect at least part of reflected light returning from the head portion in response to emission of the light. The signal processor generates brain activity data indicating a state of a brain of the user based on the image signal and calculates reliability of the brain activity data based on at least one selected from the group consisting of the image signal and a sensor signal output from a sensor that detects a change in an environment surrounding the user, the change affecting the brain activity data, and outputs reliability data indicating the reliability.

### Item 19

In the biometric measurement apparatus according to item 18, the signal processor may output the reliability data together with the brain activity data.

### Item 20

In the biometric measurement apparatus according to one of items 18 and 19, the signal processor may calculate a first value based on at least one selected from the group consisting of the image signal and the sensor signal and calculate the reliability that is lower as the first value is farther from a preset value.

### Item 21

In the biometric measurement apparatus according to one of items 18 and 19, the signal processor may calculate a first value based on at least one selected from the group consisting of the image signal and the sensor signal and calculate the reliability that is lower as a time period throughout which the first value exceeds a preset value is longer.

### Item 22

In the biometric measurement apparatus according to one of items 18 and 19, the signal processor may calculate a first value based on at least one selected from the group consisting of the image signal and the sensor signal and calculate the reliability that is lower as a time period throughout which the first value exceeds a preset value is longer within a constant time period.

### Item 23

In the biometric measurement apparatus according to one of items 18 through 22, the sensor may include at least one selected from the group consisting of a steering angle sensor, a gear position sensor, and a speed sensor, installed in a vehicle to be driven by the user.

### Item 24

In the biometric measurement apparatus according to one of items 18 through 23, the light may be light pulse. The controller may cause the image sensor to output as the image signal a second signal that is obtained by detecting a component included in a reflected light pulse prior to a start of a decrease of an intensity of the reflected light pulse returning from the head portion in response to emission of the light pulse. The signal processor may calculate the reliability based on the second signal.

### Item 25

In the biometric measurement apparatus according to one of items 1 and 2, the controller may cause the light source to repeatedly emit the light during a specific time period and cause the image sensor to repeatedly output the image signal during the specific time period. The signal processor may calculate a first value based on at least one selected from the group consisting of the image signal and the sensor signal and stop outputting the brain activity data during a time period extending from an end of a delay time following a start of a first time period to an end of the first time period within the specific time period, the first value satisfying a preset condition during the first time period.

### Item 26

A biometric measurement method according to item 26 includes causing a light source to emit light onto a head portion of a user, causing an image sensor to output an image signal by causing the image sensor to detect at least part of reflected light returning from the head portion in response to emission of the light, generating brain activity data indicating a state of a brain of the user based on the image signal, and stopping outputting the brain activity data based on at least one selected from the group consisting of the image signal and a sensor signal output from a sensor that detects a change in an environment surrounding the user, the change affecting the brain activity data.

### Item 27

A biometric measurement method according to item 27 includes causing a light source to emit light onto a head portion of a user, causing an image sensor to output an image signal by causing the image sensor to detect at least part of reflected light returning from the head portion in response to emission of the light, generating brain activity data indicating a state of a brain of the user based on the image signal, and calculating reliability of the brain activity data based on at least one selected from the group consisting of the image signal and a sensor signal output from a sensor that detects a change in an environment surrounding the user, the change affecting the brain activity data, and outputting reliability data indicating the reliability.

### Item 28

A program according to item 28 is used in a biometric measurement apparatus including a light source that emits light onto a target portion of a user, an image sensor, a controller that controls the light source and the image sensor, and a signal processor that processes a signal output from the image sensor. The controller causes the light source to emit the light and causes the image sensor to output an image signal by causing the image sensor to detect at least part of light returning from the target portion in response to the emission of the light. The program causes the signal processor to generate biometric measurement data indicating a state of the user based on the image signal. Based on the image signal an/or a sensor signal from a sensor that detects a change, affecting the biometric measurement data, in the environment surrounding the user, the program causes the signal processor to determine whether to output the biometric measurement data or, based on the sensor signal, the program causes the signal processor to calculate reliability of the biometric measurement data and output reliability data indicating the reliability.

The embodiments described below represents a comprehensive or specific example of the disclosure. Numerical values, shapes, materials, elements, and mounting locations of the elements in the embodiments are described for exemplary purposes only, and are not intended to limit the disclosure. Elements not described in independent claims indicative of the broadest concept, from among the elements of the embodiments, may be any elements.

In accordance with the present disclosure, some or all of circuits, units, apparatuses, members, or modules, or some or all of functional blocks of block diagrams may be implemented by using one or more electronic circuits including a semiconductor device, a semiconductor integrated circuit (IC), or a large scale integration (LSI) chip. The LSI chip or the IC chip may be integrated into a single chip or multiple chips. The functional blocks excluding the memories may be integrated into a single chip. The LSI chip and IC chip are quoted herein, but the LSI chip may be referred to as a system LSI chip, a very large scale integration (VLSI) chip, or an ultra large scale integration (ULSI) chip, depending on a difference in the degree of integration. A field programmable gate array (FPGA) or a reconfigurable logic device may be used for the same purposes. The FPGA is programmable after the manufacture of the LSI chip. The reconfigurable logic device is reconfigurable in terms of the connection and configuration inside the LSI chip.

The functions or operations of some or all of circuits, units, apparatuses, members, or modules may be implemented by using software. In this case, the software may be recorded on non-transitory recording media including one or more of read-only memories (ROMs), optical disks, and hard disk drives. When a processor executes the software, a function identified by the software is performed by the processor or a peripheral device thereof. A system or apparatus may include one or more non-transitory recording media having recorded the software thereon, a processor, and a hardware device in use, such as an interface.

A biometric measurement apparatus of the embodiment is specifically described with reference to the drawings.

### First Embodiment

### 1. Biometric Measurement Apparatus

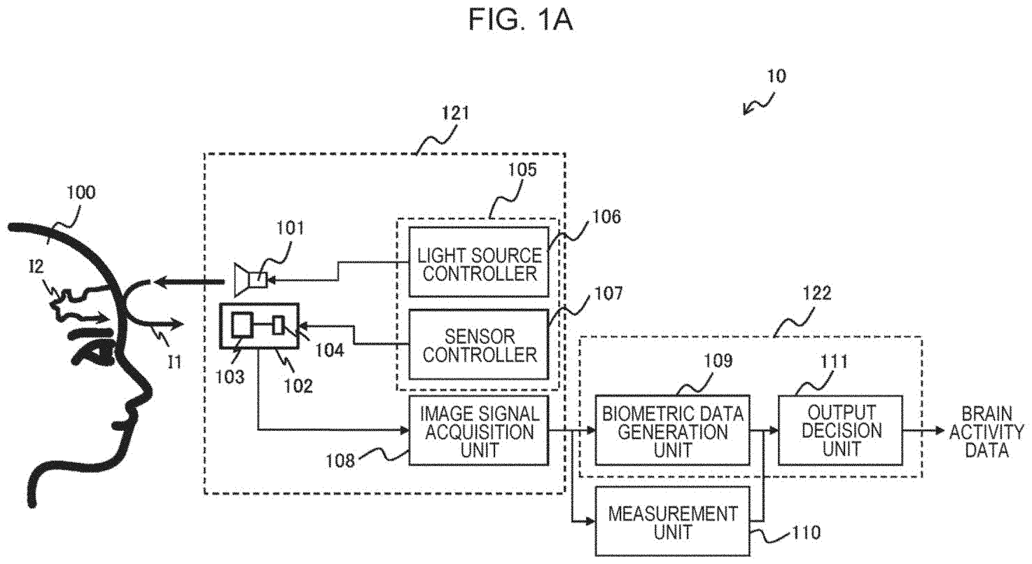

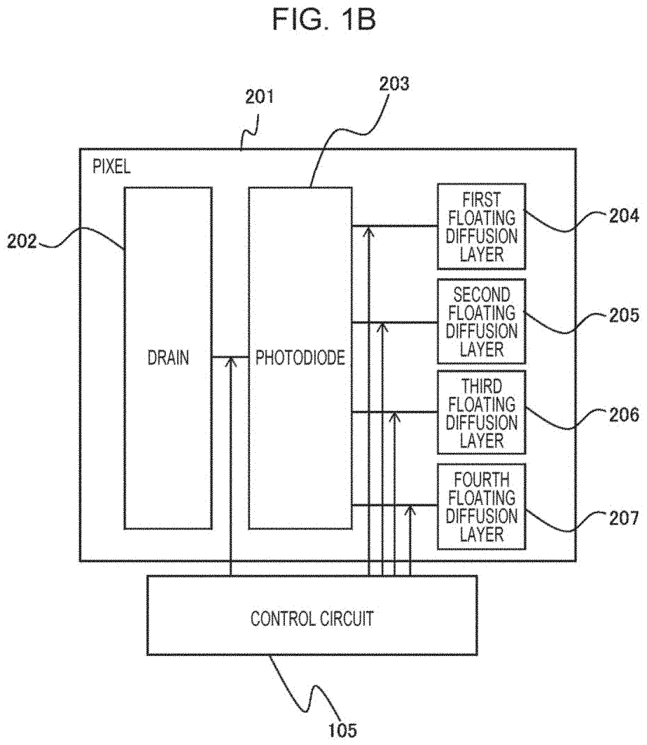

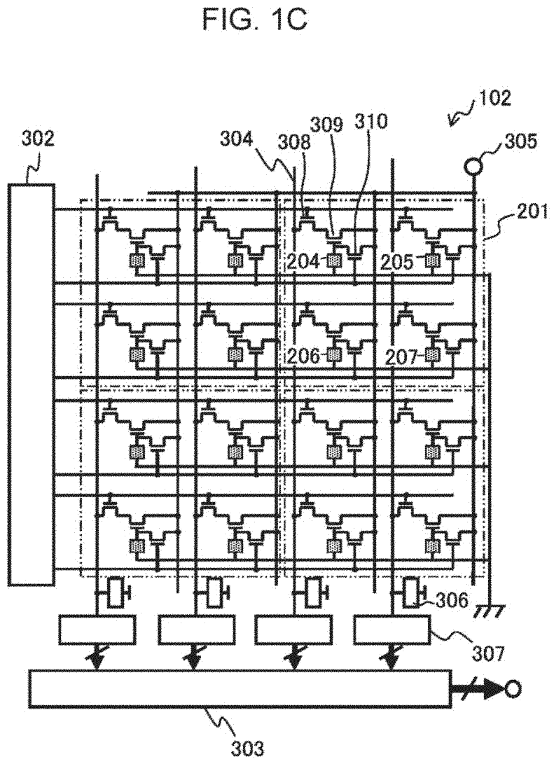

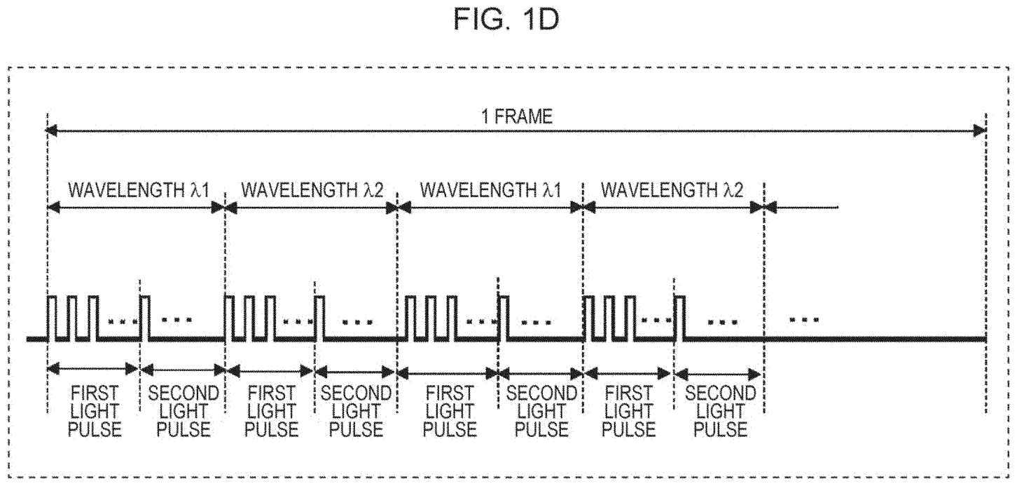

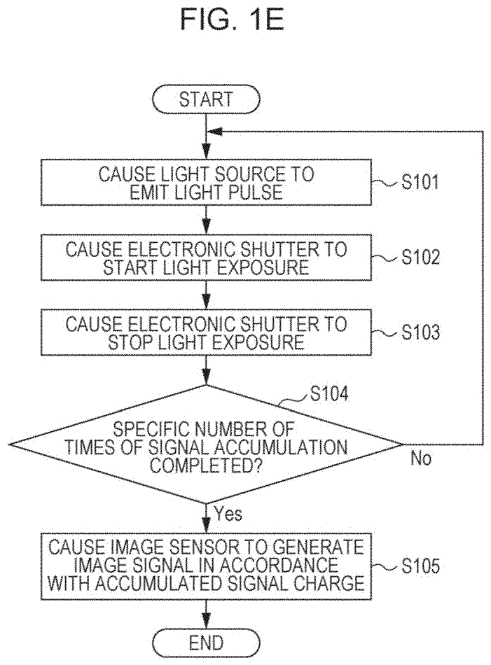

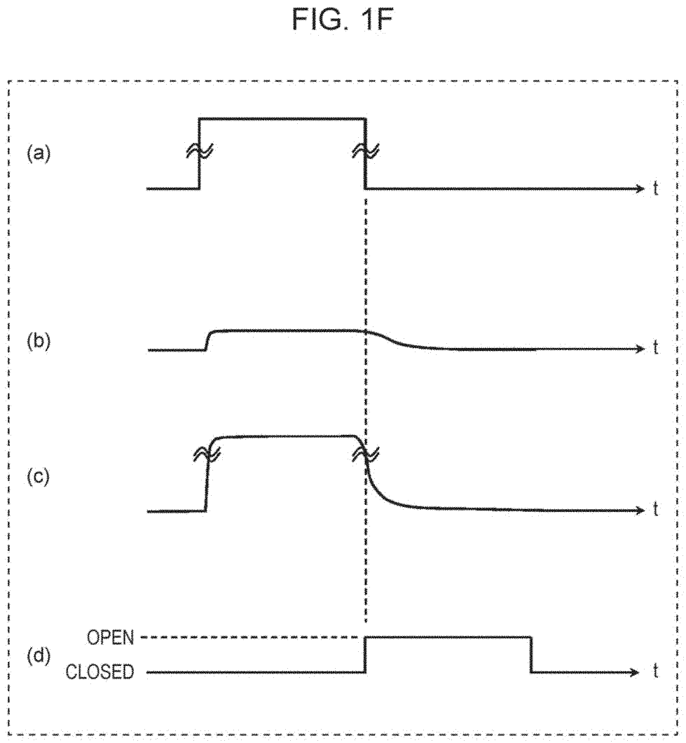

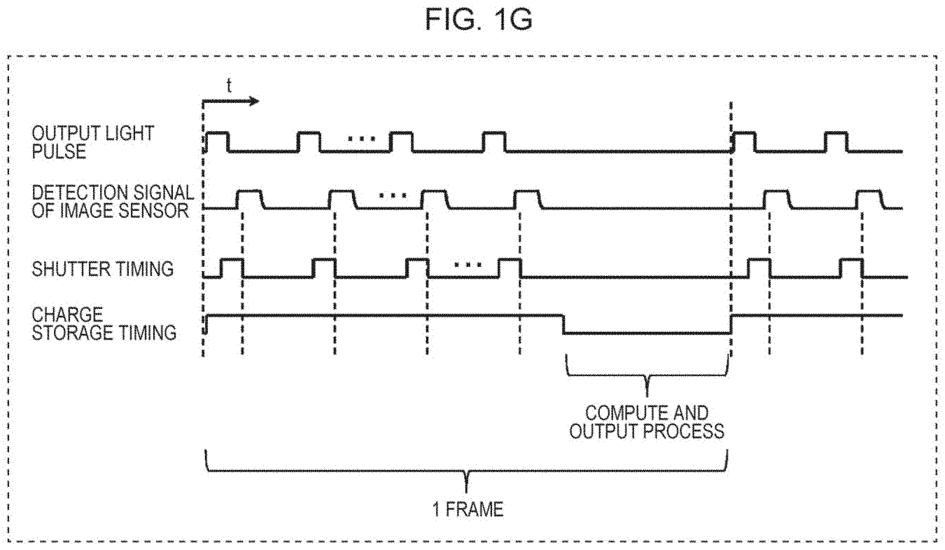

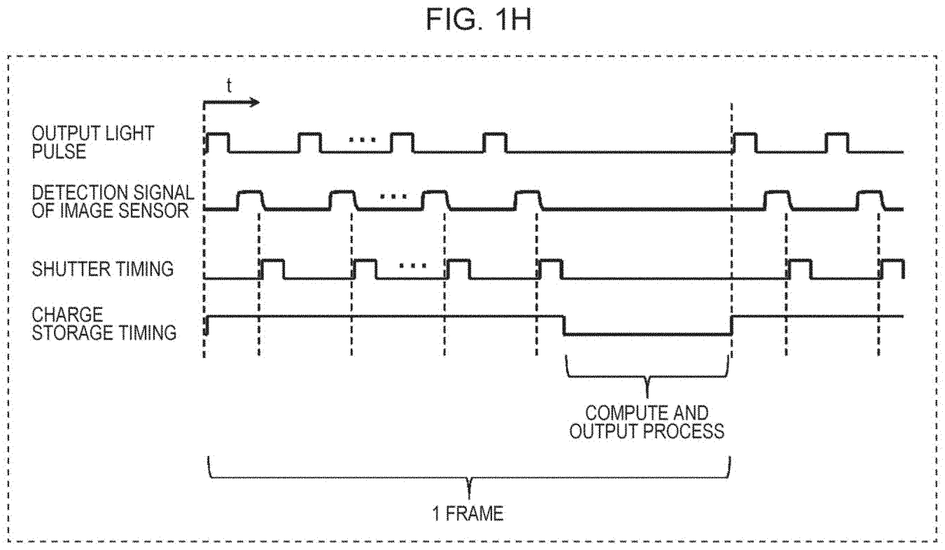

The configuration and process of the biometric measurement apparatus 10 of a first embodiment are described with reference to FIG. 1A through FIG. 3.

FIG. 1A schematically illustrates the biometric measurement apparatus 10 of the first embodiment. The biometric measurement apparatus 10 includes an imaging unit 121, measurement unit 110, and signal processing unit 122. The imaging unit 121 includes a light source 101, image sensor 102 including a photoelectric converter 103 and charge storage unit 104, control circuit 105 including a light source controller 106 and sensor controller 107, and image signal acquisition unit 108. The signal processing unit 122 includes a biometric data generation unit 109 and output decision unit 111.

### 1.1 Light Source 101

The light source 101 emits light to an target portion of a user 100. For example, the target portion of the user 100 is the head of the user 100 and specifically is the forehead of the user 100. Light emitted from the light source 101 and reaching the user 100 is split into a surface reflection component I1 reflected from the surface of the user 100 and an internal scatter component I2 scattered inside the user 100. The internal scatter component I2 is a component that is reflected once, or scattered or multi-scattered inside the body. Light is emitted to the forehead of the user 100 and reaches a location at a depth of 8 mm to 16 mm inside from the surface of the forehead, such as the brain, and some light then returns back to the biometric measurement apparatus 10. This is the internal scatter component I2. The surface reflection component I1 includes three components, a direct reflection component, a diffuse reflection component, and a scatter reflection component. The direct reflection component is a reflection component that has an angle of incidence and an angle of reflection that are equal to each other. The diffuse reflection component is a component that is diffused and then reflected on surface irregularities. The scatter reflection component is a component that is scattered and then reflected at an internal tissue close to the surface. When light is emitted to the forehead of the user 100, the scatter reflection component is scattered and then reflected at the outer layer of the skin. In the context of the disclosure, the surface reflection component I1 reflected at the surface of the user 100 includes these three components. The surface reflection component I1 and the internal scatter component I2 vary in the direction of travel depending on reflection or scattering and part of the surface reflection component I1 and part of the internal scatter component I2 reach the image sensor 102.

The acquisition method of the internal scatter component I2 is described below. The light source 101 emits a light pulse repeatedly by several times at specific time intervals or specific timings in response to an instruction from the light source controller 106. The light pulse emitted from the light source 101 may be a rectangular wave having a falling edge time being almost zero. In the context of the specification, the “falling edge time” is a time period from when the decrease in the intensity of the light pulse starts to when the decrease in the intensity of the light pulse ends. Light incident on the user 100 travels via a variety of paths within the user 100 and then exits from the surface of the user 100 with a time difference. For this reason, the trailing edge of the internal scatter component I2 of the light pulse becomes broad. If the target portion is the forehead, the breadth of the trailing edge of the internal scatter component I2 is about 4 ns. In view of this, the falling edge time of the light pulse may be set to be less than or equal to half the breadth, specifically, less than or equal to 2 ns. The falling edge time may be less than or equal to 1 ns. The rising edge time of the light pulse emitted from the light source 101 may be any time. In the context of the specification, the “rising edge time” is a time period from when the increase in the intensity of the light pulse starts to when the increase in the intensity of the light pulse ends. Not the rising edge portion of the light pulse but the falling edge portion of the light pulse is used to detect the internal scatter component I2 in the first embodiment. The rising edge portion of the light pulse is used to detect the surface reflection component I1. The light source 101 may be a laser diode (LD). Light emitted from the laser has a falling edge portion substantially vertical to the time axis and has sharp time response.

The wavelength of the light emitted from the light source 101 may be any wavelength falling within a wavelength range of from 650 nm to 950 nm. The wavelength range falls within red to near infrared waves. In the context of the specification, light refers to not only visible light but also infrared light rays. The wavelength range is referred to as an “optical window for living body” and is relatively hard to be absorbed by moisture and skin in the living body. The use of light in the wavelength range may lead to a higher detection sensitivity if a living body is a detection target. In the first embodiment, when a change in the blood flow in the skin and brain of the user 100 is detected, utilized light may be considered to be absorbed by oxygenated hemoglobin (HbO₂) and deoxygenated hemoglobin (Hb). Oxygenated hemoglobin and deoxygenated hemoglobin are different in terms of the wavelength dependency on light absorption. If a change occurs in the blood flow, oxygenated hemoglobin and deoxygenated hemoglobin vary in concentration. The degree of absorbing light also varies. Therefore, if the blood flow varies, an amount of detected light varies in time.

The light source 101 may emit light rays of two or more wavelengths falling within the wavelength range described above. The light rays of multiple wavelengths may be emitted by their respective light sources.

Since the biometric measurement apparatus 10 of the first embodiment measures the user 100 in a non-contact fashion, the light source 101 used in the measurement is designed to account for the effect on the retina. For example, the light source 101 complying with the laser safety standards Class 1 standardized in each country is used. If the Class 1 is satisfied, low illuminance light with accessible emission limit (AEL) less than 1 mW is emitted to the user 100. The light source 101 does not necessarily have to meet Class 1. For example, light may be diffused or attenuated by placing a diffusion plate or a neutral density (ND) filter in front of the light source 101 to meet the laser safety standard Class.

The light pulse emitted from the light source 101 does not have to be ultrashort pulse. Any pulse length is acceptable. When light is emitted on the forehead to measure the cerebral blood flow, an amount of light of the internal scatter component I2 is substantially lower than an amount of light of the surface reflection component I1, for example, an amount of light of the internal scatter component I2 is one-thousandth to ten-thousandth of an amount of light of the surface reflection component I1. Considering the laser safety standard, the lower amount of light of the emission light presents difficulty in detecting the internal scatter component I2. If the light source 101 emits a light pulse having a relatively longer pulse length, a storage amount of the internal scatter component I2 time-delayed increases, leading to an increase in the amount of detection light and an increase in the signal to noise (SN) ratio.

The light source 101 emits a light pulse having a pulse length of 3 ns or longer. Light scattered within a living tissue, such as the brain, typically has a time expansion as long as about 4 ns.

The light source 101 may emit a light pulse having a pulse length of 5 ns or longer, or light pulse having a pulse length of 10 ns or longer. On the other hand, if the pulse length is too long, unused light increases in vain. The light source 101 is thus designed to emit a light pulse having a pulse length of 50 ns or shorter. The light source 101 may emit a light pulse having a pulse length of 30 ns or shorter and further may emit a light pulse having a pulse length of 20 ns or shorter.

The emission pattern of the light source 101 may be uniform within an irradiation area. The biometric measurement apparatus 10 of the first embodiment may reduce the surface reflection component I1 by separating the surface reflection component I1 from the internal scatter component I2 in time. The light source 101 having a uniform intensity distribution may thus be used. The emission pattern having the uniform intensity distribution may be produced by diffusing light emitted from the light source 101 with a diffusion plate.

In a way different from the related art technique, the first embodiment may detect the internal scatter component I2 exiting from a location of the user 100 that is irradiated with the light from the light source 101. By irradiating a spatially wide area of the user 100, measurement resolution may be increased.

### 1.2 Image Sensor 102

The image sensor 102 detects at least part of the reflected light pulse returned from a target portion of the user 100 that is irradiated with the light emitted from the light source 101. The image sensor 102 outputs one or more image signals responsive to an intensity of the detected light. The image signal acquisition unit 108 acquires the image signal output from the image sensor 102. The image signal acquisition unit 108 outputs the acquired image signal to the biometric data generation unit 109 and measurement unit 110 described below. If brain activity data indicating the state of the brain is generated as biometric measurement data from the image signal, the image signal is a signal responsive to an intensity of light included in at least part of the falling edge period of the reflected light pulse.

The image sensor 102 includes the photoelectric converter 103 and the charge storage unit 104. Specifically, the image sensor 102 includes multiple photodetector cells that are two-dimensionally arrayed and acquires two-dimensional information of the user 100 at a time. In the context of the specification, the photodetector cell is also referred to as a “pixel.” The image sensor 102 may be any image sensor, such as a charge-coupled device (CCD) sensor or a complementary metal oxide semiconductor (CMOS) image sensor.

The image sensor 102 includes an electronic shutter. The electronic shutter is a circuit that controls the timing of imaging. According to the first embodiment, the sensor controller 107 in the control circuit 105 has the function of the electronic shutter. The electronic shutter controls a time period for a single storage cycle during which received light is converted into an effective electrical signal and is stored and a time period during which the signal storage is suspended. The signal storage period is also referred to as an “exposure time period” or an “imaging time period.” In the discussion that follows, the length of the exposure time period is also referred to as a shutter length. A time period from the end of one cycle of the exposure time period to the start of another cycle of the exposure time period may also be referred to as a “non-exposure time period.” In the following discussion, the state of exposure is referred to as “open” and the state of non-exposure is referred to as “closed.”

The image sensor 102 may adjust the exposure time period and the non-exposure time period with the electronic shutter in steps of sub-nano seconds, for example, 30 ps to 1 ns. According to the first embodiment, if at least the brain activity data is generated, the biometric measurement apparatus 10 does not necessarily have to correct an amount of light of a subject. For this reason, the shutter length does not have to be longer than the pulse length. For example, the shutter length may be set to be a value within a range from 1 ns to 30 ns. The biometric measurement apparatus 10 of the first embodiment may reduce the shutter length. This may reduce the effect of a dark current included in the detection signal.

If information on the cerebral blood flow is detected by emitting light onto the forehead of the user 100, the attenuation rate of the light inside the user 100 is substantially large. The exit light may be attenuated to one-millionth of the level of the corresponding incident light. If the emission of one pulse alone may be insufficient to in terms of the amount of light to detect the internal scatter component I2. The emission in the laser safety standard Class 1 results in a weak amount of light. In such a case, the light source 101 emits the light pulse several times and the image sensor 102 performs an exposure operation several times with the electronic shutter, accordingly. The detection signals are thus accumulated, increasing sensitivity.

The configuration of the image sensor 102 is described below.

The image sensor 102 includes multiple pixels two-dimensionally arranged on an imaging screen. Each pixel may include a photoelectric conversion element, such as a photodiode, and one or more charge storage units. In the following discussion, each pixel includes a photoelectric conversion element and two charge storage units. The photoelectric conversion element generates a signal charge responsive to an amount of light received through photoelectric conversion. One of the two charge storage units stores a signal charge caused by the surface reflection component I1of the light pulse and the other of the two charge storage units stores a signal charge caused by the internal scatter component I2 of the light pulse.

The control circuit 105 causes the light source 101 to emit one or more light pulses to acquire the internal scatter component I2. The control circuit 105 causes each pixel of the image sensor 102 to detect a component included within the falling edge period of each light pulse returned from the target portion of the user 100. The component includes the internal scatter component I2. The control circuit 105 causes the image sensor 102 to output a signal resulting from the detection. According to the first embodiment, the image signal based on the internal scatter component I2 is used to generate the biometric measurement data. The light source 101 may emit light rays of two wavelengths.

The control circuit 105 causes the light source 101 to emit one or more light pulses to acquire the surface reflection component I1. The control circuit 105 causes each pixel of the image sensor 102 to detect a component included prior to the falling edge period of each light pulse returned from the target portion of the user 100. The component includes the surface reflection component I1. The phrase “prior to the falling edge period” refers to before the start of the decrease of the intensity of each light pulse. The control circuit 105 causes the image sensor 102 to output a signal resulting from the detection. If a component included prior the falling edge period of each light pulse is detected, the SN ratio of the image signal may be increased. In the context of the specification, the detection of the component included prior to the falling edge period of each light pulse refers to the case in which a component included in the rising edge period of each light pulse is detected or the case in which the whole of each pulse is detected. According to the first embodiment, the image signal based on the surface reflection component I1 is used to measure a state related to the validity of the biometric measurement data.

FIG. 1B illustrates a configuration of a pixel 201 of the image sensor 102. FIG. 1B schematically illustrates the pixel 201 and does not necessarily denote the actual structure of the pixel 201. The pixel 201 in FIG. 1B includes a photodiode 203 performing photoelectric conversion, a first floating diffusion layer 204, a second floating diffusion layer 205, a third floating diffusion layer 206, a fourth floating diffusion layer 207, each floating layer serving as the charge storage unit, and a drain 202 discharging the signal charge.

Photons entering each pixel in response to the exit of one light pulse is converted into signal electrons as a signal charge by the photodiode 203. In response to a control signal from the control circuit 105, the resulting signal electrons are discharged to the drain 202 or sorted to one of the first floating diffusion layer 204 through the fourth floating diffusion layer 207.

The emission of the light pulse from the light source 101, storage of the signal charge to the first floating diffusion layer 204, the second floating diffusion layer 205, the third floating diffusion layer 206, and the fourth floating diffusion layer 207, and discharging of the signal charge to the drain 202 are repeated in that order. The repetitive operation is performed fast. For example, the repetition operation is repeated tens of thousands of times to hundreds of millions of times within the time of one frame of a video. For example, the time of one frame is about 1/30 second. Finally, the pixel 201 generates and outputs four image signals in response to the signal charges respectively stored on the first floating diffusion layer 204 through the fourth floating diffusion layer 207.

The control circuit 105 causes the light source 101 to repeatedly emit a light pulse having a wavelength λ1 and a light pulse having a wavelength λ2. The state of the user 100 may be analyzed by selecting as the wavelength λ1 and the wavelength λ2 two wavelengths having different absorption rates to the internal tissues of the user 100. For example, a wavelength longer than 805 nm may be selected as the wavelength λ1 and a wavelength shorter than 805 nm may be selected as the wavelength λ2. In this way, changes in the oxygenated hemoglobin concentration and deoxygenated hemoglobin concentration of the blood of the user 100 may be detected.

The control circuit 105 first emits the light pulse of the wavelength λ1 to the light source 101. The control circuit 105 causes the signal charge to be stored at the first floating diffusion layer 204 during a first time period while the internal scatter component I2 of the light pulse of the wavelength λ1 is incident on the photodiode 203. The light pulse is emitted at a predetermined timing to acquire the internal scatter component I2. The light pulse is referred to as a first light pulse.

In succession, the control circuit 105 causes the signal charge to be stored at the second floating diffusion layer 205 during a second time period while the surface reflection component I1 of the light pulse of the wavelength λ1 is incident on the photodiode 203. The light pulse is emitted at a predetermined timing different from the timing for the acquisition of the surface reflection component I1 to acquire the surface reflection component I1. The light pulse is referred to as a second light pulse.

The control circuit 105 causes the light source 101 to emit the light pulse of the wavelength λ2. The control circuit 105 causes the signal charge to be stored at the third floating diffusion layer 206 during a third time period while the internal scatter component I2 of the wavelength λ2 is incident on the photodiode 203.

In succession, the control circuit 105 causes the signal charge to be stored at the fourth floating diffusion layer 207 during a fourth time period while the surface reflection component I1 of the light pulse of the wavelength λ2 is incident on the photodiode 203.

When a specific period of time has elapsed since the start of the emission of the light pulse of the wavelength λ1, the control circuit 105 causes the first floating diffusion layer 204 and the second floating diffusion layer 205 to successively store the signal charges from the photodiode 203. When the specific period of time has elapsed since the start of the emission of the light pulse of the wavelength λ2, the control circuit 105 causes the third floating diffusion layer 206 and the fourth floating diffusion layer 207 to successively store the signal charges from the photodiode 203. This operation is repeated multiple times. To an amount of estimate disturbance light and ambient light, a time period may be organized to store the signal charge at another floating diffusion layer (not illustrated) with the light source 101 turned off. A signal free from the disturbance light and ambient light may be obtained by subtracting a signal charge amount at the other floating diffusion layer from the signal charge amount at the first floating diffusion layer 204 through the fourth floating diffusion layer 207.

According to the first embodiment, the number of charge storage units are four. The number of charge storage units may be set to be two or more depending on purposes. For example, if a single wavelength only is used, the number of charge storage units may be two. If a single wavelength only is used and the surface reflection component I1 is not detected, the number of charge storage units is one per pixel. Even when two or more wavelengths are used, imaging by using different wavelength may be performed in different frame. In this case, the number of charge storage units may be one. As described below, if the detection of the surface reflection component I1 and the detection of the internal scatter component I2 are performed in different frames, the number of charge storage units may be one.

FIG. 1C illustrates an example of the configuration of the image sensor 102. Referring to FIG. 1C, a region enclosed by a two-dot chain line box denotes a single pixel 201. The pixel 201 includes a photodiode. FIG. 1C illustrates only 4 pixels in two rows and two columns. The image sensor 102 actually includes many more pixels. The pixel 201 includes as the four floating diffusion layers the first floating diffusion layer 204 through the fourth floating diffusion layer 207. Signals stores at the four floating diffusion layers, namely the first floating diffusion layer 204 through the fourth floating diffusion layer 207, are handled as signals from four pixels in a standard CMOS image sensor and output from the image sensor 102.

The pixel 201 includes four signal detector circuits. The signal detector circuits include a source follower transistor 309, a row selection transistor 308, and a reset transistor 310. In this case, the reset transistor 310 corresponds to the drain 202 in FIG. 1B, and a pulse input to the gate of the reset transistor 310 corresponds to a drain discharge pulse. Each transistor includes but is not limited to a field-effect transistor formed on a semiconductor substrate. Referring to FIG. 1C, one of the input terminal and the output terminal of the source follower transistor 309 is connected to one of the input terminal and the output terminal of the row selection transistor 308. The one of the input terminal and the output terminal of the source follower transistor 309 is typically a source. The one of the input terminal and the output terminal of the row selection transistor 308 is typically a drain. The gate of the source follower transistor 309 serving as a control terminal is connected to the photodiode 203. The signal charges as holes or electrons generated by the photodiode 203 are stored at a floating diffusion layer serving as a charge storage unit between the photodiode 203 and the source follower transistor 309.

The first floating diffusion layer 204 through the fourth floating diffusion layer 207 are connected to the photodiode 203 though the connection thereof is not illustrated in FIG. 1C. A switch may be connected between the photodiode 203 and each of the first floating diffusion layer 204 through the fourth floating diffusion layer 207. The switches switch the conduction state between the photodiode 203 and each of the first floating diffusion layer 204 through the fourth floating diffusion layer 207 in response to the signal storage pulse from the control circuit 105. In this way, the start and stop of the storage of the signal charge at each of the first floating diffusion layer 204 through the fourth floating diffusion layer 207 is controlled. The electronic shutter of the first embodiment has a mechanism for such exposure control.

The signal charges stored at the first floating diffusion layer 204 through the fourth floating diffusion layer 207 are read by turning on the gate of the row selection transistor 308 via a row selection circuit 302. A current flowing from a source follower power source 305 to the source follower transistor 309 and a source follower load 306 is amplified in response to a signal voltage of the first floating diffusion layer 204 through the fourth floating diffusion layer 207. This analog signal responsive to the current and read via a column signal line 304 is digital-to-analog converted into digital signal data by an analog-digital converter circuit 307 connected on a per column basis. The digital signal data is read column by column by a column selection circuit 303 and output by the image sensor 102. The row selection circuit 302 and the column selection circuit 303 read one row, next row, and so on to read information on the signal charge at the floating diffusion layers of all the rows. After reading all the signal charges, the control circuit 105 resets all the floating diffusion layers by turning on the gates of the reset transistors 310. In this way, the imaging of one frame is complete. Similarly, by repeating high-speed imaging of the frame, the image sensor 102 completes the imaging of a series of frames.

According to the first embodiment, the CMOS type image sensor 102 is used. The image sensor 102 may be a single photon counting type element or an amplifying type CCD, such as electron multiplying CCD (EMCCD) or intensified CCD (ICCD).

FIG. 1D schematically illustrates an example of a process in a frame of the first embodiment. Referring to FIG. 1D, the emission of the light pulse of the wavelength λ1 may alternate with the emission of the light pulse of the wavelength λ2 by several times within one frame as illustrated in FIG. 1D. In this way, a time difference between the acquisition timings of detection images by two wavelengths may be reduced and the imaging operations by using the light pulses of two wavelengths are thus performed substantially at the same time.

According to the first embodiment, the image sensor 102 detects both the surface reflection component I1 and the internal scatter component I2 of the light pulse. The biometric measurement data on the user 100 may be generated from a time or spatial change in the internal scatter component I2. On the other hand, data related to the validity of the biometric measurement data is measured from a time or spatial change in the surface reflection component I1.

In the context of the specification, a signal related to the validity of the biometric measurement data is also referred to as an “validity signal.”

### 1.3 Control Circuit 105

The control circuit 105 controls the process of the light source 101 and the image sensor 102. Specifically, the control circuit 105 adjusts a time difference between the emission timing of the pulsed light of the light source 101 and the shutter timing of the image sensor 102. The time difference may also be hereinafter referred to as a “phase” or a “phase delay.” The “emission timing” of the light pulse of the light source 101 indicates the timing at which the rising of the light pulse emitted from the light source 101 starts. The control circuit 105 may adjust the phase by varying the emission timing or the shutter timing.

The control circuit 105 may be designed to remove an offset component from the signal detected by a photoreceptor of the image sensor 102. The offset component is a signal component from environment light, such as sun light or fluorescent light, or disturbance light. The offset component of the environment light or disturbance light may be estimated by the image sensor 102 that detects a signal with the light source 101 turned off in no light emission state.

The control circuit 105 may be an integrated circuit including a processor, such as a central processing unit (CPU) or microcomputer, and a memory. By executing a program stored on the memory, the control circuit 105 adjusts the emission timing and the shutter timing, estimates the offset component, and removes the offset component.

FIG. 1E is a flowchart illustrating a process of the light source 101 and the image sensor 102, controlled by the control circuit 105. The control circuit 105 includes the light source controller 106 and the sensor controller 107 and performs a process illustrated in FIG. 1G described below. In the process described herein, only the internal scatter component I2 is detected.

In step S101, the light source controller 106 causes the light source 101 to emit the light pulses during a specific time period. The electronic shutter of the image sensor 102 remains closed stopping exposure. The sensor controller 107 causes the electronic shutter to stop exposure until the end of a time period used for a portion of the light pulse reflected from the surface of the user 100 to reach the image sensor 102. In step S102, the sensor controller 107 causes the electronic shutter to start light exposure at the timing at which another portion of the light pulse scattered within the user 100 reaches the image sensor 102. A specific period of time later, the sensor controller 107 causes the electronic shutter to stop light exposure in step S103. In step S104, the control circuit 105 determines whether the number of cycles of signal storage performed reaches a specific value. If the no path is followed, the operations in steps S101 through S103 are repeated until the yes path is followed. If the yes path is followed in step S104, the sensor controller 107 causes the image sensor 102 to generate and output a signal indicating an image based on a signal charge stored at each floating diffusion layer in step S105.

Through the above process, the light component scattered within the measurement target may be detected at a higher accuracy level. The emission and exposure are performed once or performed more times as appropriate.

### 1.4 Measurement Unit 110

The measurement unit 110 measures the validity signal indicating the validity of the biometric measurement data and transfers measurement results as a signal to the output decision unit 111.

According to the first embodiment, the validity signal is measured from an image including a time or spatial change in the surface reflection component I1. The image is acquired from the image sensor 102.

The measurement unit 110 may include an arithmetic circuit performing an arithmetic process, such as image processing. The arithmetic circuit is implemented by a combination of a programmable logic device (PLD), such as a digital signal processor (DSP) or a field programmable gate array (FPGA), or a central processing unit (CPU) or a graphic processing unit (GPU), and a computer program.

In another embodiment described below, the validity signal may be measured from an image including a time or spatial change in the internal scatter component I2.

In yet another embodiment described below, the validity signal may be measured from an image other than the image output from the image sensor 102. In this case, the measurement unit 110 may include a sensor internal to or external to the biometric measurement apparatus 10. The sensor measures a change in the environment surrounding the user 100 and outputs the sensor signal indicating the change. The sensor signal may be a signal indicating an amount of change in the physical change affecting the biometric measurement data out of the physical change in the surrounding environment. The sensor signal may be a signal indicating a specific state of the surrounding environment affecting the biometric measurement data. The phrase “affecting the biometric measurement data” is intended to mean the intrusion of noise into the biometric measurement data or a state that the biometric measurement data is unmeasurable. The sensor may be at least one selected from a group of an illuminance sensor, an acceleration sensor, a speed sensor, a steering angle sensor, and a gear position sensor.

### 1.5 Signal Processing Unit 122

The signal processing unit 122 processes a signal output from the image sensor 102. The signal processing unit 122 includes the biometric data generation unit 109 and the output decision unit 111. Referring to FIG. 1A, the signal processing unit 122 and the measurement unit 110 are separate units but they may be integrated into a single unitary unit.

The biometric data generation unit 109 generates the biometric measurement data on the user 100 in accordance with an image signal output from the image sensor 102. If the biometric measurement data is the brain activity data on the user 100, the biometric data generation unit 109 generates video data indicating a time change in the cerebral blood flow by processing the image signal including a time or spatial change in the internal scatter component I2, and transfers a signal responsive to the generated video data to the output decision unit 111. For example, the time change in the cerebral blood flow is a time change in the oxygenated hemoglobin concentration and/or the deoxygenated hemoglobin concentration.

If the biometric measurement data is the brain activity data on the user 100, the biometric data generation unit 109 may generate not only video data on the cerebral blood flow but also other data related to the cerebral blood flow. For example, the data related to the cerebral blood flow may be the psychological state of the user 100 estimated from the video data on the cerebral blood flow.

It is known that a change in the cerebral blood flow or a component in the blood, such as hemoglobin, is closely related to the nervous activity of human. For example, the cerebral blood flow or the component in the blood flow changes in response to a change in the feeling of a human. The psychological state of the user 100 may thus be estimated by measuring biometric information, such as the cerebral blood flow or the change in the component in the blood flow. For example, the psychological state of the user 100 may be related to the mood, feeling, state of health, or sensitivity to temperature of the user 100. For example, the mood may be comfortability or uncomfortability to the user 100. The feeling may be relief, anxiety, sorrow, or indignation. The state of health is energy or lethargy. The sensitivity to temperature may be hot, cold, or humid. An index indicating the degree of brain activity may also fall within the psychological state. The index may be related to skill level, proficiency level, and concentration. In the context of the specification, data related to the cerebral blood flow is collectively referred to as the brain activity data.

Based on measurement results sent from the measurement unit 110, the output decision unit 111 determines whether to output the biometric measurement data sent from the biometric data generation unit 109. The measurement results include a value that the measurement unit 110 has calculated in accordance with the image signal output from the image sensor 102 and/or the sensor signal output from the sensor.

When the output of the biometric measurement data is stopped, the output decision unit 111 output a signal indicating that the biometric measurement data is invalid at that stop timing. The output decision unit 111 continues to output the same data as the biometric measurement data that is valid at the immediately preceding moment. When the biometric measurement data becomes valid again, the output decision unit 111 output data interpolated from the biometric measurement data valid at the immediately preceding moment.

Like the measurement unit 110, the signal processing unit 122 may include an arithmetic circuit performing an arithmetic process, such as image processing. The arithmetic circuit is implemented by a combination of a programmable logic device (PLD), such as a digital signal processor (DSP) or a field programmable gate array (FPGA), or a central processing unit (CPU) or a graphic processing unit (GPU), and a computer program. The signal processing unit 122 and the control circuit 105 may be physically separate circuits or may be integrated into a unitary circuit. The signal processing unit 122 may be an element of an external device, such as a remotely installed server. In such a case, the external device, such as a server, transmits or receives data to or from the light source 101, the image sensor 102, and the control circuit 105.

### 1.6 Other Elements

The biometric measurement apparatus 10 may include an image focusing optical system that forms a two-dimensional (2D) image of the user 100 on a photoreceptor surface of the image sensor 102. The optical axis of the image focusing optical system is substantially perpendicular to the photoreceptor surface of the image sensor 102. The image focusing optical system may include a zoom lens. By changing the location of the zoom lens, the image focusing optical system changes an expansion rate of the 2D image of the user 100. In this way, the resolution of the 2D image on the image sensor 102 changes. The user 100 even spaced apart the biometric measurement apparatus 10 may closely observe the 2D image by expanding a desired measurement region.

The biometric measurement apparatus 10 include between the user 100 and the image sensor 102 a bandpass filter that allows light in a wavelength band emitted from the light source 101 or only light close to the wavelength band to pass therethrough. In this way, the influence of a disturbance component, such as ambient light, may thus be reduced. The bandpass filter may include a multi-layer film or an absorption filter. The bandwidth of the bandpass filter may have a width of from 20 to 100 nm taking into consideration the temperature of the light source 101 and a band shift due to oblique light incidence on the filter.

The biometric measurement apparatus 10 may include a polarizer between the light source 101 and the user 100 and another polarizer between the image sensor 102 and the user 100. The polarization direction of the polarizer arranged on the light source 101 and the polarization direction of the polarizer arranged on the image sensor 102 satisfies the crossed Nicols condition. In this way, this arrangement may preclude a regular reflection component of the surface reflection component I1 of the user 100 having angles of incidence and reflection equal to each other from reaching the image sensor 102. In other words, an amount of light of the surface reflection component I1 reaching the image sensor 102 may be reduced.

### 2. Process of Light Source and Image Sensor

The biometric measurement apparatus 10 of the first embodiment may separately detect the surface reflection component I1 and the internal scatter component I2. If the user 100 is a human and the target portion is the forehead, the desired signal intensity responsive to the internal scatter component I2 is relatively very small. As previously described, this is because the amount of emission light satisfying the laser safety standard is relatively very small and light scattered and absorbed by the scalp, cerebrospinal fluid, skull, gray matter, white matter and blood flow is relatively large. The magnitude of a change in the signal intensity responsive to the blood flow or a change in a component in the blood flow during brain activity is several tenths of the magnitude of the signal intensity prior to the change and is thus relatively very small. According to the first embodiment, the surface reflection component I1 that is thousands to tens of thousands of the signal component to be desired is removed as much as possible during the imaging.

The process of the light source 101 and the image sensor 102 in the biometric measurement apparatus 10 of the first embodiment is described below.

Referring to FIG. 1A, the surface reflection component I1 and the internal scatter component I2 are generated when the light source 101 emits the light pulse to the user 100. A portion of each of the surface reflection component I1 and the internal scatter component I2 reaches the image sensor 102. The internal scatter component I2 is transmitted through the user 100 while the light pulse is emitted from the light source 101 and reaches the image sensor 102. The optical path length of the internal scatter component I2 thus becomes longer than the optical path length of the surface reflection component I1. The time of arrival of the internal scatter component I2 at the image sensor 102 is typically later than the time of arrival of the surface reflection component I1 at the image sensor 102.

FIG. 1F schematically illustrates an optical signal that arrives at the image sensor 102 when a rectangular light pulse emitted from the light source 101 is returned from the user 100. The horizontal axis denotes time t in signals (a) through (d). The vertical axis denotes intensity in the signals (a) through (c) and represents open or closed state of the electronic shutter in the signal (d). The signal (a) denotes the surface reflection component I1. The signal (b) denotes the internal scatter component I2. The signal (c) denotes the sum of the surface reflection component I1 of the signal (a) and the internal scatter component I2 of the signal (b). As illustrated in the signal (a), the surface reflection component I1 remains in a rectangular shape. As illustrated in the signal (b), the internal scatter component I2 is the sum of light rays having traveled along a variety of optical path lengths. For this reason, the signal (b) is the light pulse with an extended trailing slope. In other words, since the falling edge period of the internal scatter component I2 is longer than the falling edge period of the surface reflection component I1, the ratio of the internal scatter component I2 to the surface reflection component I1 to be extracted is higher in the optical signal in the signal (c). As illustrated in the signal (d), the electronic shutter performs the exposure operation at the trailing edge of the surface reflection component I1 and later. The trailing edge or later represents the timing when the surface reflection component 11 falls and a period subsequent to the timing. The shutter timing of the electronic shutter is adjusted by the control circuit 105. As previously described, the biometric measurement apparatus 10 of the first embodiment detects separately the surface reflection component I1 and the internal scatter component I2 that has penetrated a deeper portion of the target portion. The emission pulse length and the shutter length have any length. Unlike a method of using a related art streak camera, the biometric measurement apparatus 10 features a simple mechanism with reduced cost.

Referring to the signal (a) in FIG. 1F, the trailing edge of the surface reflection component I1 vertically falls. In other words, the time from the start of the falling to the end of the falling of the surface reflection component I1 is zero. Actually, however, the falling edge waveform of the light pulse emitted from the light source 101 may not completely be vertical, the surface of the user 100 may have fine irregularities, and/or scattering may occur on the outer layer of the skin of the user 100. In such a case, the trailing edge of the surface reflection component I1 does not fall vertically. Since the user 100 is opaque, an amount of light of the surface reflection component I1 is substantially larger than an amount of light of the internal scatter component I2. Even when the falling edge of the surface reflection component I1 is slightly sloped rather than vertically falls, the internal scatter component I2 may be buried in the trailing edge of the surface reflection component I1. During the reading period of the electronic shutter, a time delay may occur by electron transfer. A binary reading as in the signal (d) in FIG. 1F may be difficult to achieve. The control circuit 105 may thus delay the shutter timing of the electronic shutter slightly from the falling edge of the surface reflection component I1. For example, the shutter timing of the electronic shutter may be delayed by about 0.5 to 5 ns. Instead of adjusting the shutter timing of the electronic shutter, the control circuit 105 may adjust the emission timing of the light source 101. The control circuit 105 adjusts a time difference between the shutter timing of the electronic shutter and the emission timing of the light source 101. When the blood flow or the change in the component in the blood flow is measured in a non-contact fashion during the brain activity, the originally smaller surface reflection component I1 is even more reduced if the shutter timing is delayed too much. In view of this, the shutter timing is may be set close to the trailing edge of the surface reflection component I1. The time delay due to the scattering in the user 100 is 4 ns. The maximum delay time of the shutter timing is thus as long as about 4 ns.

The image sensor 102 may be exposed to each of the light pulses emitted from the light source 101 at the shutter timing of the same phase. In this way, a detection amount of light of the internal scatter component I2 is amplified.

In addition to or instead of using the bandpass filter between the user 100 and the image sensor 102, the control circuit 105 may be used to estimate an offset component by imaging during the same exposure time with no light emitted from the light source 101. The offset component estimated is removed from the signal detected by each pixel of the image sensor 102. In this way, a dark current component generated on the image sensor 102 is removed.

The internal scatter component I2 includes the internal information on the user 100, such as cerebral blood flow information. An amount of light absorbed by blood varies depending on a time variation in the cerebral blood flow of the user 100. As a result, the detection amount of light of the image sensor 102 increases or decreases accordingly. By monitoring the internal scatter component I2, the brain activity state may be estimated in view of a change in the cerebral blood flow of the user 100. According to the first embodiment, a signal indicating the internal scatter component I2 out of signals output from the image sensor 102 is referred to as a “brain activity signal.” The brain activity signal may include increase/decrease information on the cerebral blood flow of the user 100.

The detection method of the surface reflection component I1 is described below. The surface reflection component I1 includes surface information on the user 100. For example, the surface information is blood flow information on the face and scalp of the user 100. When the light pulse is emitted from the light source 101 and arrives at the user 100, the image sensor 102 detects the surface reflection component I1 out of optical signals that return to the image sensor 102.

FIG. 1G schematically illustrates an example of a timing chart to detect the surface reflection component I1. In order to detect the surface reflection component I1, the electronic shutter may be opened before the light pulse arrives at the image sensor 102 and closed before the trailing edge of the light pulse arrives at the image sensor 102 as illustrated in FIG. 1G. The intrusion of the internal scatter component I2 may be reduced by controlling the electronic shutter in this way. As a result, the percentage of light passing through the vicinity of the surface of the user 100 is increased. The shutter closing timing may be set to be immediately subsequent to the arrival of the light to the image sensor 102. This may lead to the signal detection with a higher percentage of the surface reflection component I1 having a relatively shorter optical path length. The pulse of the user 100 or the oxygenation of facial blood flow of the user 100 may be detected by acquiring a signal of the surface reflection component I1. As another acquisition method of the surface reflection component I1, the image sensor 102 may detect the whole light pulse emitted from the light source 101 or continuous light emitted from the light source 101.

FIG. 1H schematically illustrates an example of a timing chart to detect the internal scatter component I2. The signal of the internal scatter component I2 may be acquired by opening the electronic shutter during a time period while the trailing edge of the light pulse arrives at the image sensor 102.

The surface reflection component I1 may be detected by a device other than the biometric measurement apparatus 10 that acquires the internal scatter component I2. The device other than the biometric measurement apparatus 10 that acquires the internal scatter component I2 may be a pulse wave meter or Doppler blood flow meter. The other device is used by accounting for inter-device timing synchronization, light interference, and arrangement of detection locations. If time division imaging is performed using the same camera or the same sensor in the first embodiment, a displacement in time and space is difficult to occur. When both the signal of the surface reflection component I1 and the signal of the internal scatter component I2 are acquired with the same sensor, the component to be acquired may be alternated frame by frame as illustrated in FIG. 1G and FIG. 1H. Alternatively, as described with reference to FIG. 1B through FIG. 1D, the components acquired fast within one frame are alternately switched. In such a case, a detection time difference between the surface reflection component I1 and the internal scatter component I2 may be reduced. Both the signal of the surface reflection component I1 and the signal of the internal scatter component I2 may be acquired from the same light pulse.

Each of the signal of the surface reflection component I1 and the signal of the internal scatter component I2 may be acquired by using light of two wavelengths. For example, light pulses of two wavelengths 750 nm and 850 nm may be used. In this way, a concentration change in the oxygenated hemoglobin and a concentration change in the deoxygenated hemoglobin may be calculated from changes in the detection amounts of light on the two wavelengths. When each of the surface reflection component I1 and the internal scatter component I2 is acquired by using the two wavelengths, a method of switching four types of charge storage at a high speed within one frame as described with reference to FIG. 1B through FIG. 1D may be used. This method may reduce a time lag in the detection signal.

The biometric measurement apparatus 10 emits infrared light or visible light in the form of pulse toward the forehead of the user 100 and detects the pulse or a change in an amount of oxygenated hemoglobin in the scalp or the forehead by referring to a time change in the surface reflection component I1. The light source 101 emits infrared light or visible light to acquire the surface reflection component I1. The measurement is possible by using the infrared light pulse day and night. The visible light providing a higher sensitivity may be used to measure the pulse. If the amount of light is insufficient, a dedicated light source may be used for reinforcement. The internal scatter component I2 includes a light component that has reached the brain. The time increase or decrease in the cerebral blood flow may be measured by measuring a time change in the internal scatter component I2.

Light having reached the brain is transmitted through the scalp and the surface of the face. The variation in the blood flow of the scalp and the face is also detected together. The effect of the variation is to be removed or reduced. When the brain activity data is used as the biometric measurement data, the biometric data generation unit 109 may perform a subtraction operation subtracting the surface reflection component I1 from the internal scatter component I2 detected by the image sensor 102. In this way, cerebral blood flow information free from blood flow information on the scalp and face may thus be acquired. In the subtraction operation, a value resulting from multiplying the surface reflection component I1 by a coefficient that is 1 or a greater value determined in view of the optical path length is subtracted from the signal of the internal scatter component I2. Based on the mean of optical constants of heads of ordinary persons, the coefficient is determined by using simulation or experiment. The subtraction operation may be more easily performed by using the light of the same wavelength on the same camera or the same sensor. This is because a displacement in time and space may be easily reduced and it is easy to cause the characteristics of a scalp blood flow component included in the internal scatter component I2 to match the characteristics of the surface reflection component I1.

The skull is present between the brain and the scalp. The 2D distribution of the cerebral blood flow is independent of the 2D of the blood flow of the scalp and face. In accordance with the signal detected by the image sensor 102, the 2D distribution of the internal scatter component I2 and the 2D distribution of the surface reflection component I1 may be separated by using a statistical technique, such as independent component analysis or principal component analysis.

### 3. Sequence of Biometric Measurement

A method of measuring the biometric measurement data on the user 100 by using the biometric measurement apparatus 10 is described below. According to the first embodiment, the measurement unit 110 measures a body motion of the user 100 from an image signal including the surface reflection component I1. According to the first embodiment, the body motion refers to a distance of motion of the target portion. The target portion is the head of the user 100. The distance of motion is a displacement from a reference position. According to the first embodiment, based on the measurement results, the output decision unit 111 determines whether to output the biometric measurement data.

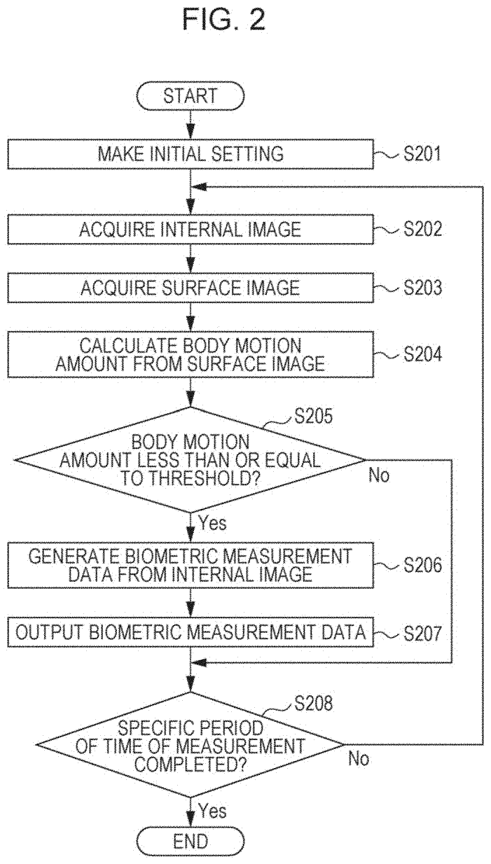

FIG. 2 is a flowchart illustrating a process of measuring the biometric measurement data on the user 100 in accordance with the first embodiment.

In step S201, the biometric measurement apparatus 10 makes an initial setting before the measurement of the biometric measurement data. The operation in step S201 includes a sub-step in which the control circuit 105 appropriately adjusts the emission timing of the light pulse from the light source 101 and the shutter timing of the image sensor 102 in view of the distance between the biometric measurement apparatus 10 and the user 100. The operation in step S201 also includes a sub-step in which the control circuit 105 causes the image signal acquisition unit 108 to transfer the image signal including the surface reflection component I1 to the measurement unit 110 and a sub-step in which the measurement unit 110 calculates the location of the head in the initial state and stores information on the location of the head on a memory (not illustrated) in the measurement unit 110.

In step S202, the control circuit 105 causes the image signal acquisition unit 108 to transfer to the biometric data generation unit 109 the image signal representing an internal image including the internal scatter component I2.

In step S203, the control circuit 105 causes the image signal acquisition unit 108 to transfer to the measurement unit 110 the image signal representing a surface image including the surface reflection component I1.

The order of the operations in steps S202 and S203 may be reversed.

In step S204, the measurement unit 110 calculates the location of the target portion from the image signal representing the surface image and calculates a difference between the location of the target portion and the location of the target portion in the initial state stored on the memory. The measurement unit 110 thus calculates the body motion. The difference is an amount of displacement of the target portion. To calculate the location of the target portion from the image signal, a related art technique, such as feature point extraction technique of using edge detection, may be used. A 2D image of the face includes high spatial-frequency components in larger amount than a 2D image of the cerebral blood flow. The 2D image of the face is more beneficial in that the extraction of the feature point is easy. The measurement unit 110 transfers to the output decision unit 111 the body motion as a difference with respect to the reference in the initial setting.

In step S205, the output decision unit 111 determines whether the body motion is lower than or equal to a threshold. The threshold falls within a range of from 1 to 30 mm. When the 2D distribution of the cerebral blood flow is acquired as the biometric measurement data and if a desired resolution of the 2D distribution is relatively low, the threshold may be set to be a higher value.

If the body motion is lower than or equal to the threshold, the biometric data generation unit 109 generates the biometric measurement data from the image signal representing the internal image and transfers the biometric measurement data to the output decision unit 111 in step S206. In step S207, the output decision unit 111 outputs the biometric measurement data. The output may be displayed on a display (not illustrated) in the biometric measurement apparatus 10. The output may be used for control by a host system (not illustrated) higher than the biometric measurement apparatus 10.

If the body motion exceeds the threshold, steps S206 and S207 are skipped. The output decision unit 111 stops outputting the biometric measurement data. Instead of stopping the biometric measurement data, the output decision unit 111 may output, at that timing, a signal indicating that the biometric measurement data is invalid. Alternatively, the output decision unit 111 may continue to output the same data as the biometric measurement data valid at the immediately preceding moment. When the biometric measurement data becomes valid again, the output decision unit 111 may output data that is interpolated from the biometric measurement data valid at the immediately preceding moment.

In step S208, a determination as to whether the measurement has been performed for a specific period of time. This determination may be performed by the biometric measurement apparatus 10 or the host system to which the output of the biometric measurement apparatus 10 has been supplied.

The “specific period of time” may be a time period extending until the estimation of the psychological state of the user 100 becomes available. The “specific period of time” may also be a time period extending until a series of tasks given to the user 100 are complete. The “specific period of time” may also be a time period extending until the user 100 has completed a series of operations. The series of operations may be driving an automobile or operating a game machine.

If the measurement is not complete by the end of the specific period of time, the biometric measurement apparatus 10 repeats the sequence of steps S202 through S208. If the measurement is complete by the end of the specific period of time, the biometric measurement apparatus 10 completes the measurement process.

The process of the biometric measurement apparatus 10 of the first embodiment is summarized as below. The control circuit 105 causes the light source 101 to emit light and causes the image sensor 102 to output the image signal. In response to the image signal, the biometric data generation unit 109 generates the biometric measurement data. The output decision unit 111 determines whether to output the biometric measurement data. The control circuit 105, the biometric data generation unit 109, and the output decision unit 111 repeat the operations thereof. The output decision unit 111 stops outputting the biometric measurement data during a time period while a value calculated in accordance with the image signal output from the image sensor 102 satisfies a preset condition. In the above example, the value calculated in accordance with the image signal output from the image sensor 102 is a difference indicating the displacement of the target portion calculated by the measurement unit 110. The preset condition is that the difference exceeds the threshold.

4. Process during Biometric Measurement

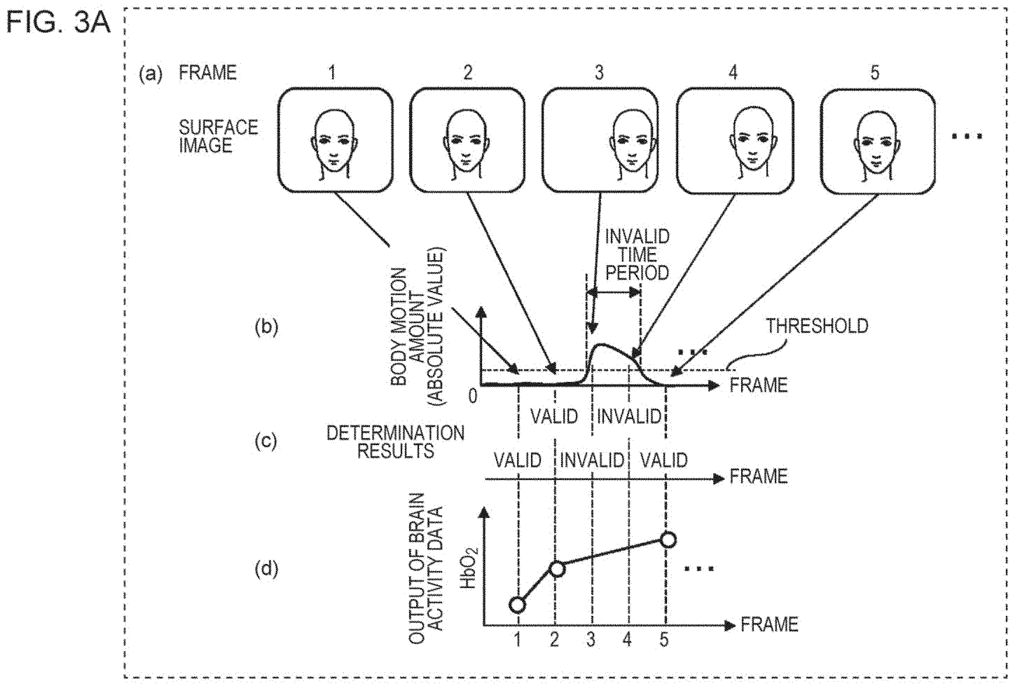

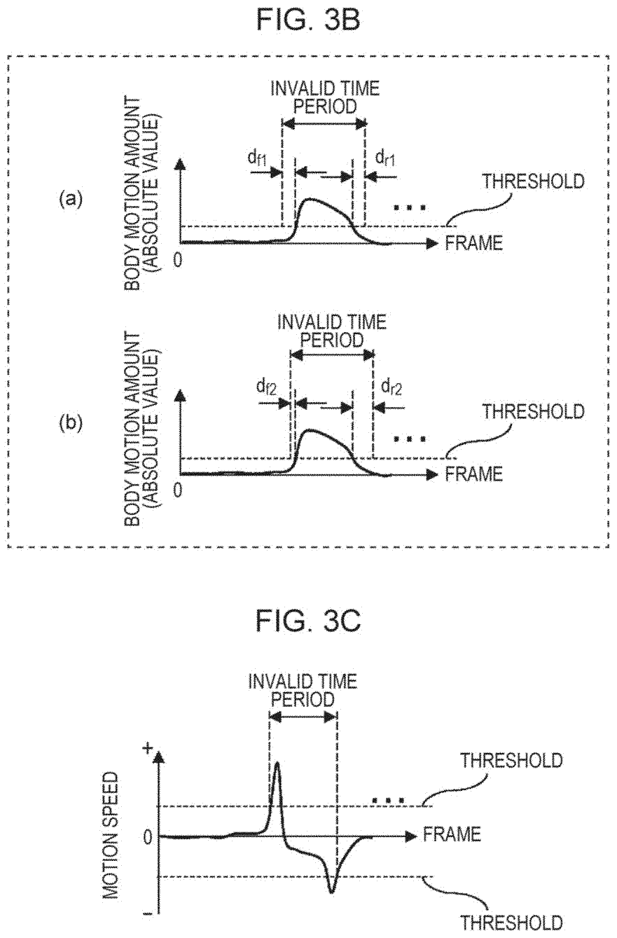

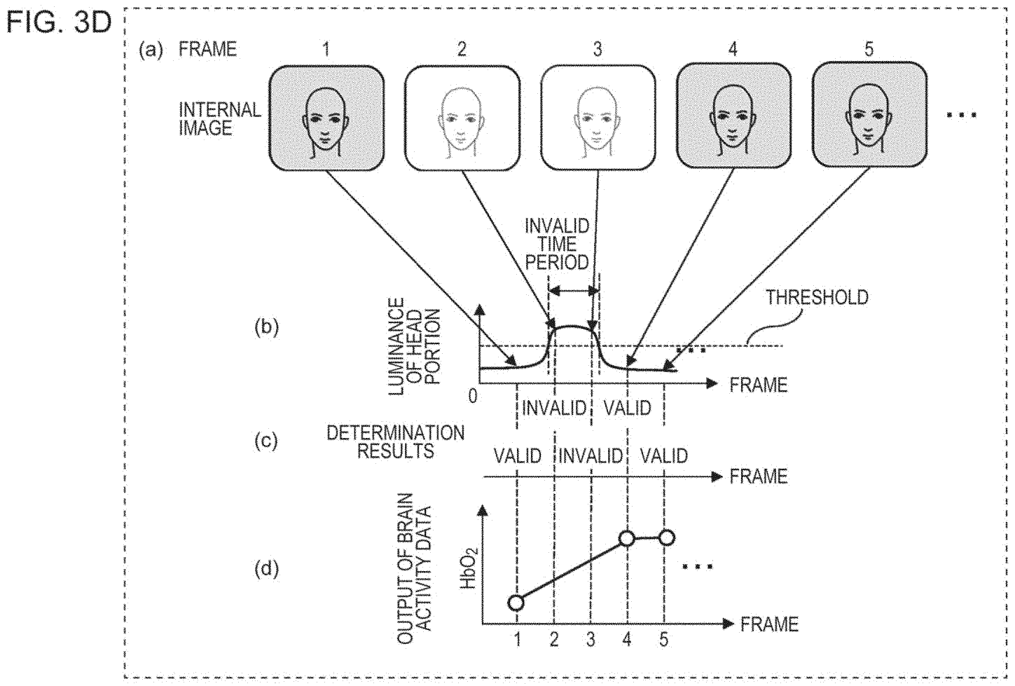

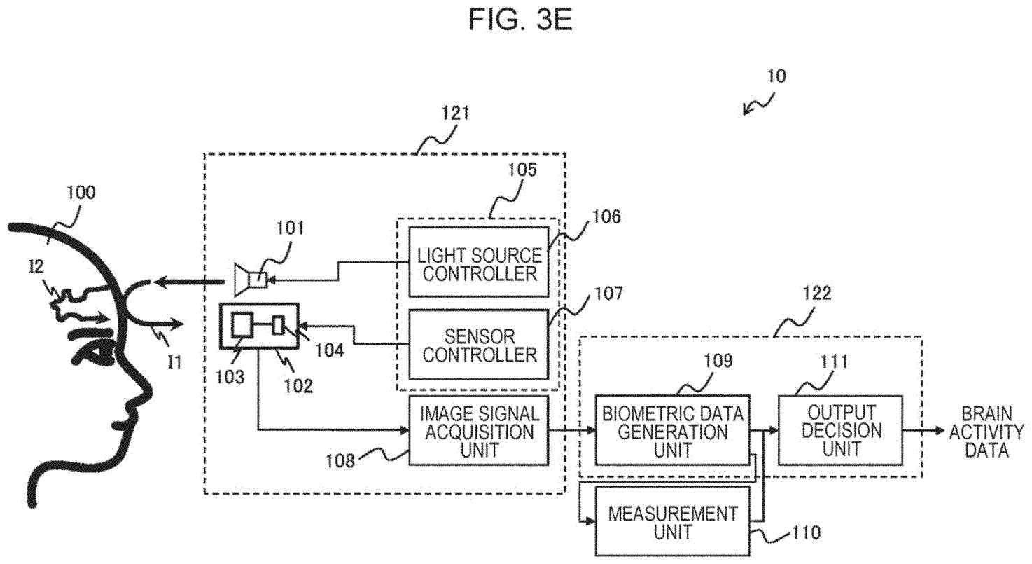

FIG. 3A illustrates a process example that measures the biometric measurement data on the user 100 in accordance with the first embodiment. In the process in FIG. 3A, the target portion is the head of the user 100.

In part (a) through part (d) of FIG. 3A, the horizontal axis denotes transition of frame or time. Part (a) of FIG. 3A schematically illustrates a change in the surface image including the surface reflection component I1. Part (b) of FIG. 3A illustrates a change in the body motion. Part (c) of FIG. 3A illustrates determination results. Part (d) of FIG. 3A illustrates a change in the oxygenated hemoglobin concentration in the forehead region as the brain activity data serving as an example of the biometric measurement data.

Each frame is numbered. One or more frames may further be inserted between two adjacent frames. The frame rate or the frequency of measurement may be within a range of from 1 frame per second (fps) to 30 fps. The frequency of measurement of the body motion may be different from the frequency of generation of the biometric measurement data. The cerebral blood flow may gently change during 1 second to several seconds. The body motion acquired as an image in a non-contact fashion changes at a speed higher than the cerebral blood flow. To detect the body motion more accurately, the frequency of measurement of the body motion may be higher than or equal to the frequency of generation of the biometric measurement data.

Referring to part (a) of FIG. 3A, the body motion measured by the measurement unit 110 exceeds the threshold in response to the motion of the target portion between frame 3 and frame 4. The body motion is correlated with the irregularity of the generated biometric measurement data. During a time period throughout which the body motion exceeds the threshold, the output decision unit 111 determines that the biometric measurement data is invalid and stops outputting the biometric measurement data. As a result, referring to part (d) of FIG. 3A, the brain activity data serving as the biometric measurement data is output in frame 1, frame 2, and frame 5. Referring to part (b) of FIG. 3A, the time period throughout which the body motion exceeds the threshold is labeled “invalid time period.”

According to the first embodiment, the biometric measurement data may be measured in a non-contact fashion without prompting the user 100 to measure again. In this way, the biometric measurement apparatus 10 capable of performing biometric measurement on a daily basis may be implemented. The biometric measurement apparatus 10 is free from computing involved in the restoration of the cerebral blood flow distribution. This may lead to computing cost reductions and biometric measurement may be performed in a low-cost manner.

FIG. 3B schematically illustrates the relationship between a change in the body motion and invalid time period. In the operation illustrated in part (b) of FIG. 3A, a time period throughout which the body motion exceeds the threshold is referred to as an invalid time period. On the other hand, referring to part (a) of FIG. 3B, the invalid time period may be set to be a time period including the time period throughout which the body motion exceeds the threshold, a time duration d_{f1} before the body motion rises crossing the threshold, and a time duration do after the body motion falls crossing the threshold. Alternatively, the invalid time period may set to be the sum of the time period throughout which the body motion exceeds the threshold and one of the time durations d_{f1} and dᵣ₁. The time durations d_{f1} and do correspond to about half as long as 0.5 frame. The output decision unit 111 may suspend the outputting of the biometric measurement data during not only the time period while the value calculated in accordance with the image signal output from the image sensor 102 satisfies the preset condition but also the time duration d_{f1} prior to the time period and/or the time duration do subsequent to the time period even when the value does not satisfy the condition.

The timing of starting the invalid time period may be after the body motion exceeds the threshold. Specifically, d_{f1} may be a negative value with reference to the timing when the body motion exceeds the threshold. This is because there may be a delay time involved in system processing from when the body motion exceeds the threshold to when the invalid time period is set.

Referring to part (b) of FIG. 3B, a time duration dᵣ₂ subsequent to the time period may be set to be longer than a time duration d_{f2} prior to the time period. For example, the time duration d_{f2} corresponds to about 0.5 frame and the time duration dᵣ₂ corresponds to about 4.5 frames. The reason for this setting is described below with reference to examples.

According to the first embodiment, the body motion is calculated from the image signal including the surface reflection component I1. On the other hand, the body motion may be calculated from the image signal including the internal scatter component I2. The image signal including the internal scatter component I2 also includes information on the external shape of the target portion. The image signal including the surface reflection component I1 may result in a larger amount of detection light. The image signal including the surface reflection component I1 is more useful in terms of a signal-to-noise (SN) ratio than the image signal including the internal scatter component I2.

According to the first embodiment, the displacement of the target portion serves as a measurement target. However, the measurement target is not limited to the displacement. The measurement target may be a motion speed of the target portion. In a frame during which the absolute value of the motion speed of the target portion exceeds a threshold, the outputting of the brain activity data may be suspended. In such a frame, a body motion is likely to occur. The motion speed of the target portion is also understood as a distance of motion between the frames.

FIG. 3C schematically illustrates the relationship between the motion speed of the target portion and invalid time period. As illustrated in FIG. 3C, the invalid time period is set to a time period from when the motion speed rises crossing a positive threshold to when the motion speed rises crossing a negative threshold after falling and crossing the positive threshold and further falling and crossing the negative threshold. Alternatively, the invalid time period is set to a time period from when the motion speed falls crossing the negative threshold to when the motion speed falls crossing the positive threshold after rising and crossing the negative threshold and further rising and crossing the positive threshold.

The measurement target may not necessarily relate to the motion of the target portion but may be a luminance value of the target portion.

FIG. 3D illustrates a process example that measures the biometric measurement data on the user 100 in accordance with the first embodiment. Referring to FIG. 3D, the target portion is the head of the user 100. Referring to part (a) of FIG. 3D, the measurement unit 110 measures the luminance value of the target portion from the internal image as the image including the internal scatter component I2. A time period during which the luminance value of the target portion exceeds a threshold is set to be the invalid time period. The output decision unit 111 stops outputting the brain activity data in frame 2 and frame 3 within the invalid time period. If the luminance value of the target portion becomes closer to the saturation value thereof in the image including the internal scatter component I2, it is difficult to accurately generate the biometric measurement data. By determining the validity in accordance with the luminance value of the target portion, the biometric measurement apparatus 10 may output only more accurate biometric measurement data.

Referring to FIG. 3D, a time period throughout which the luminance value of the target portion exceeds the threshold is set to be the invalid time period. Conversely, a time period while the luminance value of the target portion is lower than the threshold may set to be the invalid time period. If the luminance value of the target portion in the image including the internal scatter component I2 is very low, the SN ratio may be insufficient. In such a case, it is difficult to generate the biometric measurement data.

The measurement target may be a change rate of the luminance value of the target portion. The outputting of the brain activity data may be stopped in a frame where the absolute value of the change rate of the luminance value exceeds a threshold. In that frame, the luminance may be considered to be too high or too lower.

The measurement unit 110 may measure from the image signal the area of a specific region of the target portion whose the biometric measurement data is to be measured. For example, the area of the specific region of the target portion is an area of the forehead of the user 100. A related-art image processing method, such as the feature extraction using the edge detection, is used to calculate the area of the forehead portion from the image signal. If the forehead portion has many hairs and the area of the forehead portion is lower than a threshold, the outputting of the biometric measurement data may be suspended. This is because a smaller area of the target portion leads to the generated biometric measurement data at a lower accuracy level.

The measurement target may be the biometric measurement data.

FIG. 3E schematically illustrates an example of the biometric measurement apparatus 10 of the first embodiment. Unlike in FIG. 1A, generation results of the biometric measurement data are also transferred to the measurement unit 110 in FIG. 3E. The measurement unit 110 measures a change rate of the biometric measurement data. The biometric measurement data is the oxygenated hemoglobin concentration or the deoxygenated hemoglobin concentration, as the brain activity data. The cerebral blood flow mildly changes during 1 to several seconds. In contrast, the body motion and a luminance change in the target portion are at a speed faster than the cerebral blood flow and affect the measurement value of the cerebral blood flow. The biometric measurement apparatus 10 may output only accurate biometric measurement data by determining the validity in accordance with the change rate of the biometric measurement data. If the absolute value of the change rate of a value calculated from the biometric measurement data exceeds a threshold, the output decision unit 111 may stop outputting the biometric measurement data.

### Second Embodiment

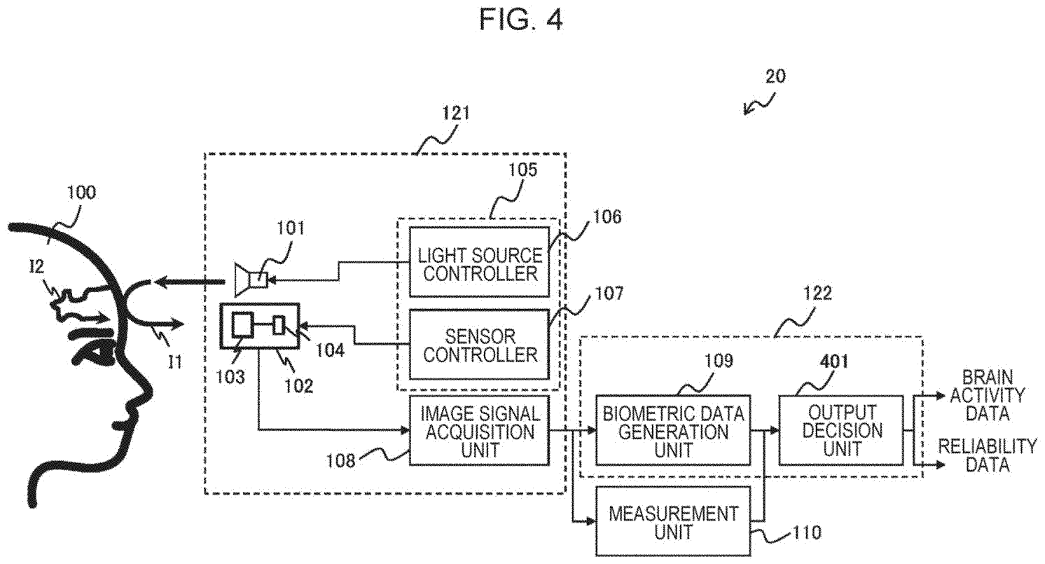

The configuration and process of a biometric measurement apparatus 20 of a second embodiment is described with reference to FIGS. 4 through 6.

FIG. 4 schematically illustrates the biometric measurement apparatus 20 of the second embodiment. According to the second embodiment, the measurement unit 110 measures the body motion of the user 100 from the image signal including the surface reflection component I1. According to the second embodiment, the body motion of the user 100 is the distance of motion of the target portion. In response to the measurement results, an output decision unit 401 calculates the reliability of the biometric measurement data and outputs reliability data, indicating the reliability, together with the biometric measurement data. Unlike the first embodiment, the output decision unit 401 in the second embodiment outputs not only the biometric measurement data but also the reliability data.

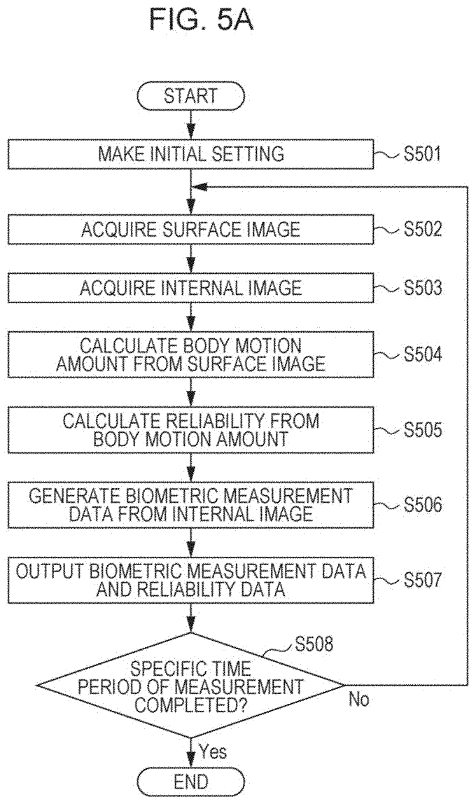

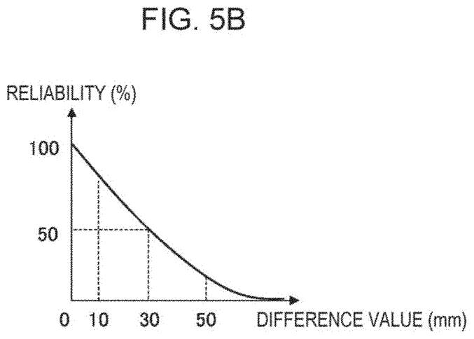

FIG. 5A is a flowchart illustrating a process example that measures the biometric measurement data on the user 100 in accordance with the second embodiment.

An initial setting in step S501, acquisition of the surface image in step S502, acquisition of the internal image in step S503, and calculation of the body motion in step S504 are respectively identical to those in steps S201 through S204 in the first embodiment.

In step S505, the output decision unit 401 calculates the reliability in response to the body motion calculated from the difference value in the initial setting.

FIG. 5B illustrates an example of relationship between the difference value and reliability. With a difference value of 0 mm, the measurement results are the most reliable. Referring to FIG. 5B, the reliability may set to be 100%, if the difference value is 0 mm. Referring to FIG. 5B, the larger the difference value, the smaller the reliability. The output decision unit 401 may calculate the reliability that is lower as the value calculated from the image signal output from the image sensor 102 is farther from a preset value.

The biometric measurement apparatus 20 of the second embodiment may output the biometric measurement data regardless of the size of the body motion. In step S506, the biometric data generation unit 109 generates the biometric measurement data from the image signal and transfers the biometric measurement data to the output decision unit 401.

In step S507, the output decision unit 401 outputs the reliability data together with the biometric measurement data. For example, the output may be displayed on a display (not illustrated) of the biometric measurement apparatus 20 or may be used to control the host system (not illustrated).

The operation is step S508 is identical to the operation in step S208 in the first embodiment.

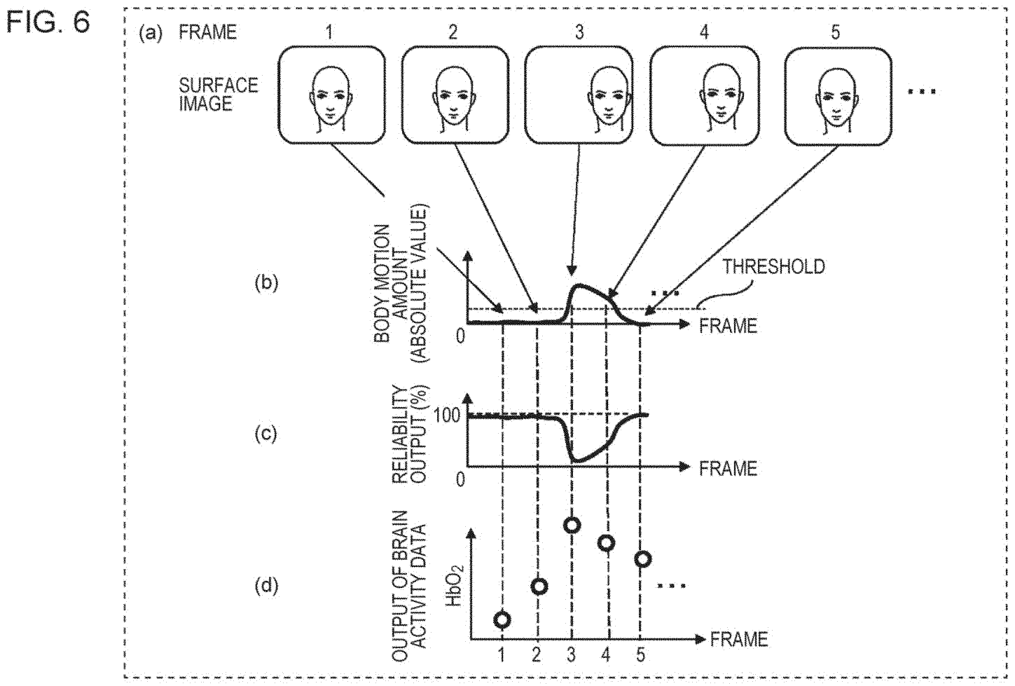

FIG. 6 illustrates a process example that measures the biometric measurement data on the user 100 in accordance with the second embodiment. Referring to FIG. 6, the target portion is the head of the user 100.

In part (a) through part (d) of FIG. 6, respectively identical to part (a) through part (d) of FIGS. 3A, the horizontal axis denotes transition of frame or time. Part (a) of FIG. 6 schematically illustrates a change in the surface image including the surface reflection component I1. Part (b) of FIG. 6 illustrates a change in the body motion. Part (c) of FIG. 6 illustrates a change in the reliability. Part (d) of FIG. 6 illustrates a change in the oxygenated hemoglobin concentration in the forehead region as the brain activity data serving as an example of the biometric measurement data.

Unlike the first embodiment, the reliability is calculated in response to the body motion of each frame in the second embodiment. Referring to part (c) of FIG. 6, the output decision unit 401 calculates the reliability that is lower as the body motion in part (b) of FIG. 6 is larger. The body motion is a difference value of the location of the target portion with respect to the initial state.

In frame 3 and frame 4, the reliability is lower due to the motion of the target portion. The output decision unit 401 may output the biometric measurement data in all the frames. The output decision unit 401 outputs the reliability data together with the biometric measurement data.

According to the second embodiment, the biometric measurement data may be measured in a non-contact fashion without prompting the user 100 to measure again. In this way, the biometric measurement apparatus 20 capable of performing biometric measurement on a daily basis may be implemented. The biometric measurement apparatus 20 is free from computing involved in the restoration of the cerebral blood flow distribution. This may lead to computing cost reductions and biometric measurement may be performed in a low-cost manner. Furthermore, beneficially, the host system may comprehensively determine the validity of the biometric measurement data using the reliability data.

According to the second embodiment, the reliability is calculated as a lower value as the measurement results of the measurement unit 110 are farther away from the threshold. The calculation method of the reliability is not limited to this method.

The reliability may not be a measurement result value but may be calculated based on the temporal trend of the measurement results. For example, as the invalid time period throughout which the measurement results remain exceeding the threshold is longer, a lower reliability may be calculated. In other words, as a time period throughout which the value calculated from the image signal output from the image sensor 102 exceeds a preset value is longer, the output decision unit 111 may calculate.

As the ratio of the invalid time period to a given time period is higher, a lower reliability may be calculated. In other words, the output decision unit 111 may calculate the reliability that is lower as a time period throughout which a value calculated from the image signal output from the image sensor 102 exceeds a preset value is longer within a constant time period. If a valid measurement value alternates with an invalid measurement value in a short time, the calculation method of the reliability described above is effective.

### Third Embodiment

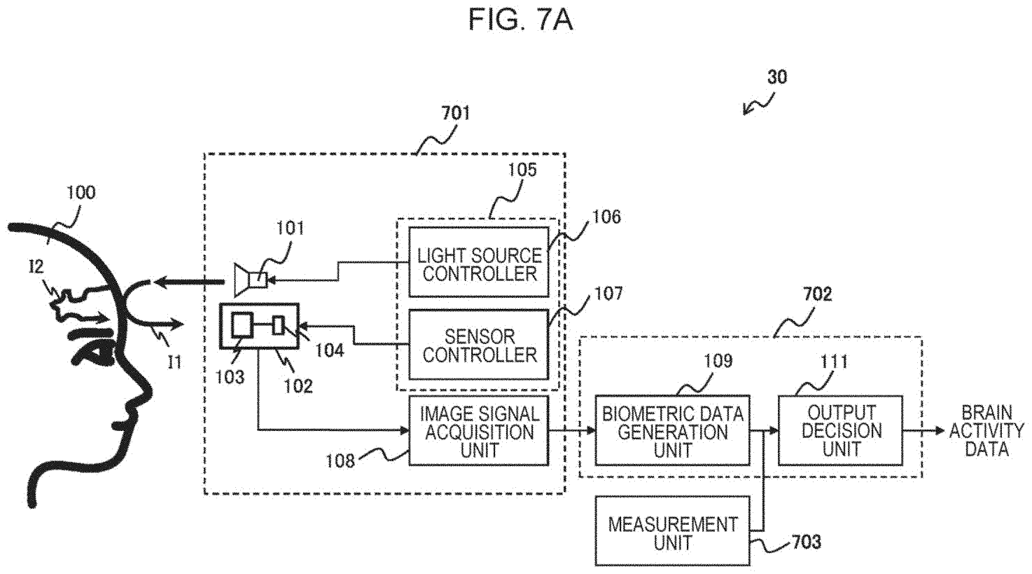

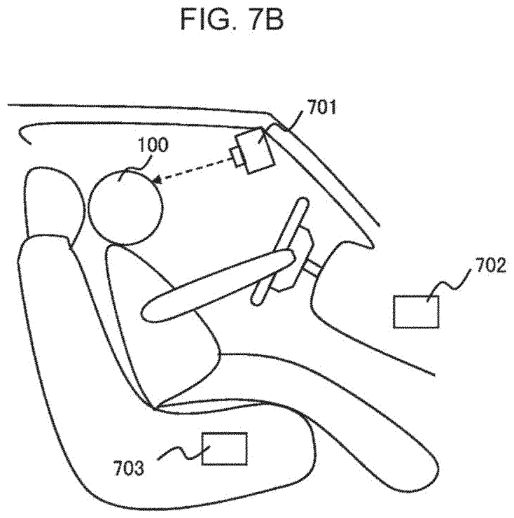

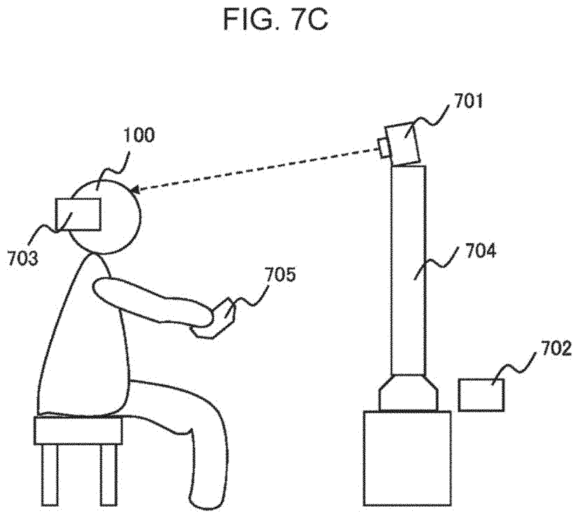

The configuration and process of a biometric measurement apparatus 30 of a third embodiment are described with reference to FIGS. 7A through 9.

FIG. 7A schematically illustrates the biometric measurement apparatus 30 of the third embodiment. The biometric measurement apparatus 30 of the third embodiment includes an imaging unit 701, signal processing unit 702, and measurement unit 703. According to the third embodiment, the measurement unit 703 includes an acceleration sensor installed in the vicinity of the user 100 in an environment surrounding the user 100 and calculates acceleration. Based on the measurement results, the output decision unit 111 determines whether to output the biometric measurement data.

FIG. 7B schematically illustrates an installation example of each element of the biometric measurement apparatus 30 in an automobile. The imaging unit 701, signal processing unit 702, and measurement unit 703 may be separately installed. The measurement unit 703 is installed in the vicinity of the user 100. The phrase “installed in the vicinity of the user 100” is intended to mean that the measurement unit 703 and the user 100 are within a distance that at least allows the body motion of the user 100 to be correlated with the acceleration measured by the measurement unit 703.

FIG. 7C schematically illustrates an installation example of each element of the biometric measurement apparatus that is mounted on a game machine or an attraction device. As in the installation example in FIG. 7B, the imaging unit 701, signal processing unit 702, and measurement unit 703 may be separately installed. In response to visual information from a display 704, the user 100 operates a controller 705. The measurement unit 703 may be mounted on a headphone or a head-mounted display worn on the head of the user 100. The measurement unit 703 measures acceleration when the target portion of the user 100 moves. The measurement unit 703 may detect a change in illuminance of the target portion in response to a change in display information of the display 704.

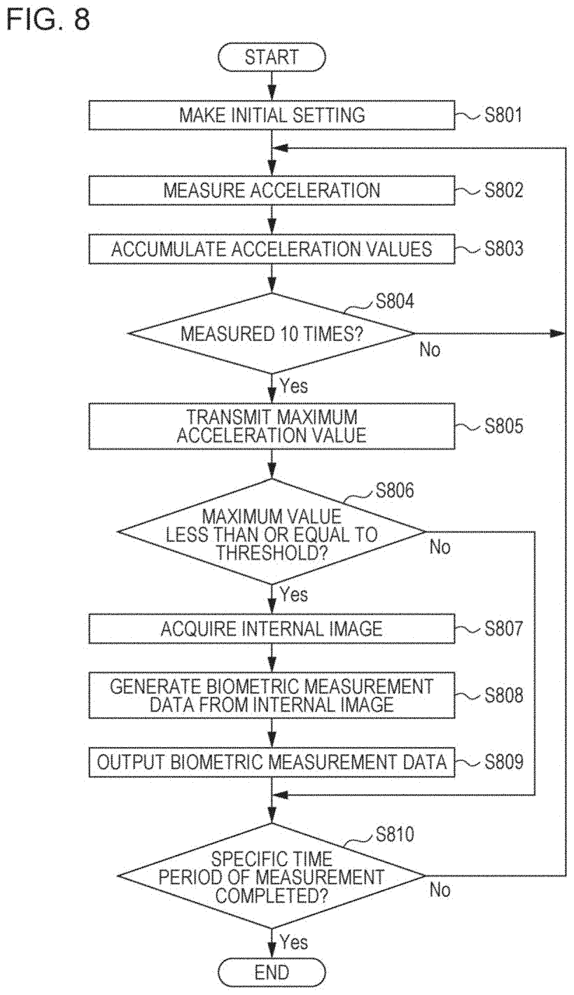

FIG. 8 is a flowchart illustrating a process example that measures the biometric measurement data on the user 100 in accordance with the third embodiment.

In step S801, the biometric measurement apparatus 30 makes an initial setting prior to measuring the biometric measurement data. As in the first embodiment, the operation in step S801 includes a sub-step in which the control circuit 105 appropriately adjusts the emission timing of the light pulse from the light source 101 and the shutter timing of the image sensor 102 in response to the distance between the biometric measurement apparatus 30 and the user 100.

In step S802, the measurement unit 703 measures acceleration. If the biometric measurement apparatus 30 is installed inside an automobile, the measurement of acceleration corresponds to the measurement of vibration of the automobile.

The frequency of measurement of acceleration may be higher than or equal to the frequency of generation of the biometric measurement data. Even a sharp vibration occurs, a high trackability of the measurement value may be achieved. As a result, the output decision unit 111 may increase the determination accuracy. According to the third embodiment, the frequency of the measurement of the acceleration may be set to be 10 times as high as the frequency of the calculation of the brain activity data.

In step S803, acceleration values are successively stored on a memory (not illustrated) in the measurement unit 703. For example, depending on the determination step of the measurement cycles of acceleration in step S804, a loop of steps S802 and S803 may be repeated 10 times. The number of measurement cycles is not limited to any number.

In step S805, the measurement unit 703 refers to the contents on the memory and transfers to the output decision unit 111 a maximum value from among 10 acceleration values stored. Instead of the maximum value, the mean may be used. According to the third embodiment, a value at least correlated with the body motion of the user 100 is calculated from 10 acceleration values.

In step S806, the output decision unit 111 determines whether the maximum value of the acceleration values received from the measurement unit 703 is lower than or equal to a threshold. For example, the threshold is an acceleration value in a fore-aft direction, left-right direction, or vertical direction and falls within a range higher than or equal to 0.1 G and lower than or equal to 1 G (G=9.8 m/s²).

In step S807, the control circuit 105 causes the image signal acquisition unit 108 to transfer the image signal including the internal scatter component I2 to the biometric data generation unit 109. Operations in steps S808, S809, and S810 are respectively identical the operations in steps S206, S207, and S208 in the first embodiment.

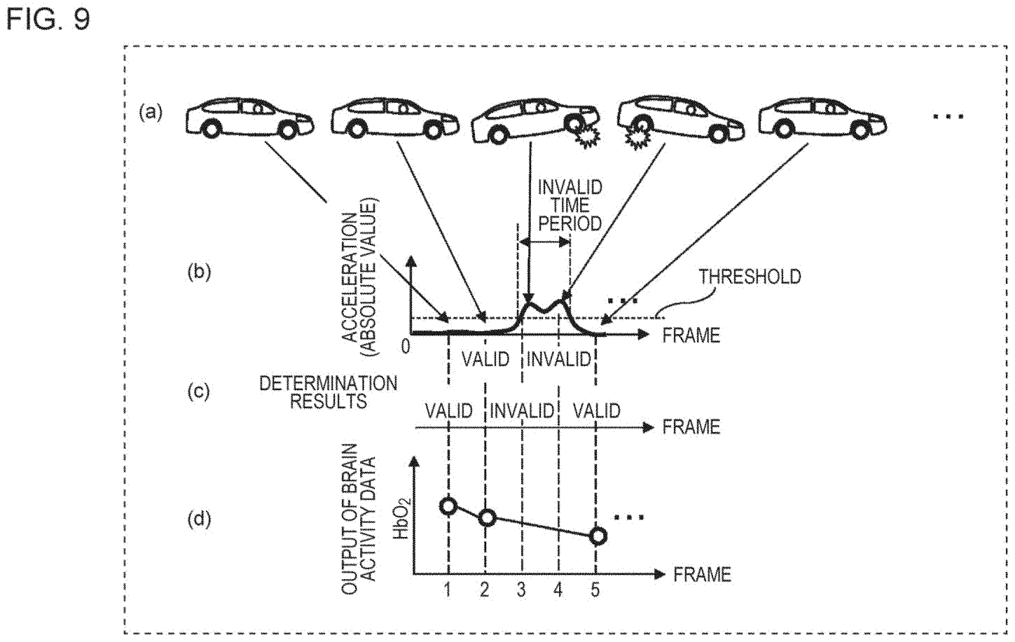

FIG. 9 illustrates a process example that measures the biometric measurement data on the user 100 in accordance with the third embodiment. Referring to FIG. 9, the target portion is the head of the user 100.

Referring to part (a) through part (d) of FIG. 9, the horizontal axis denotes the transition of frame or time. Part (a) of FIG. 9 schematically illustrates a state of a housing of an automobile or an attraction device. Part (b) of FIG. 9 illustrates a change in acceleration. Part (c) of FIG. 9 illustrates determination results. Part (d) of FIG. 9 illustrates a change in the oxygenated hemoglobin concentration in the forehead region as the brain activity data serving as an example of the biometric measurement data.

Unlike in the first embodiment, the measurement unit 703 measures acceleration in the third embodiment. Acceleration is measured since the acceleration of the environment surrounding the user 100 is correlated with the biometric measurement data on the user 100.

Referring to FIG. 9, the housings of the automobile and the attraction device vibrate in frame 3 and frame 4 and the acceleration measured by the measurement unit 703 exceeds a threshold. The body motion is correlated with the irregularity of the generated biometric measurement data. During the invalid time period throughout which the acceleration exceeds the threshold, the output decision unit 111 determines that the biometric measurement data is invalid and stops outputting the biometric measurement data. As a result, referring to part (d) of FIG. 9, the brain activity data as the biometric measurement data is output in frame 1, frame 2, and frame 5.

As described with reference to the previous embodiments, the biometric measurement data is measured in a non-contact fashion without prompting the user 100 to measure again. In this way, the biometric measurement apparatus 30 capable of performing biometric measurement on a daily basis may be implemented. The biometric measurement apparatus 30 is free from computing involved in the restoration of the cerebral blood flow distribution. This may lead to computing cost reductions and biometric measurement may be performed in a low-cost manner. Beneficially, the biometric measurement apparatus 30 is free from measuring the body motion from the image signal and thus operates with computing cost reduced.

The measurement target of the measurement unit 703 is at least correlated with the body motion and is not limited to acceleration. For example, if the biometric measurement apparatus 30 is mounted on the automobile, the measurement unit 703 may include an acceleration sensor and measure speed. The measurement unit 703 may include a steering angle sensor and measure an steering angle of the automobile.

The measurement unit 703 may include a gear position sensor and measure a gear position of the automobile. In this case, the output decision unit 111 stops outputting the biometric measurement data during a time period while the measurement results of the gear position are in a preset state. Referring to FIG. 3B, the output decision unit 111 may stop outputting during not only the time period but also a time duration prior to the start of the time period and/or a time duration subsequent to the end of the time period. For example, the preset state is a gear reverse state. In this state, the user 100 may drive looking back to back the automobile up. The target portion of the user 100 moves from the reference position, causing a body motion of the user 100.

The measurement target of the measurement unit 703 may not necessarily be related to the body motion of the user 100. The measurement unit 703 may include an illuminance sensor mounted in the vicinity of the user 100 in the surrounding environment and measure illuminance. In this case, the output decision unit 111 stops outputting the biometric measurement data during the invalid time period throughout which an illuminance value exceeds a threshold. If a luminance value of the target portion approaches the saturation value thereof in the image including the internal scatter component I2, it is difficult to accurately generate the biometric measurement data. If the illuminance value is at least correlated with the luminance value of the target portion, the validity is determined in accordance with the luminance value of the target portion. The biometric measurement apparatus 30 may thus output only more accurate biometric measurement data.

### EXAMPLES

Examples that were implemented to verify the principle of the disclosure are described below.

The biometric measurement apparatus 10 of the first embodiment in FIG. 1A was mounted in a manner such that the biometric measurement apparatus 10 faces the head of the user 100. The chin of the user 100 was placed on a chin support to control unintentional body motion. After inviting the user 100 to relax, the head of the user 100 was photographed for 30 seconds and the brain activity data was measured as the biometric measurement data.

The wavelengths of the light source were 750 nm and 850 nm. The image signal including the surface reflection component I1 was acquired at a rate of 30 fps. The image signal including the internal scatter component I2 was acquired at a rate of 5 fps. The resolution of the image signal was 320 pixels×240 pixels. An area of 50 pixels×50 pixels including the center of the forehead was set to be a target region. In the target region, spatial averaging was performed and time averaging was performed on a real-time basis. Oxygenated hemoglobin concentration was calculated based on the image signal including the internal scatter components I2 of two wavelengths. A change in the oxygenated hemoglobin concentration was monitored.

A change in the distance of motion of the face from the measurement unit 110 was also concurrently monitored in accordance with the image signal including the surface reflection component I1. Kanade-Lucas-Tomasi (KLT) algorithm was used to extract and track feature points of the face.

Only once during the measurement, the user 100 intentionally moved the head laterally and then soon settled back to the original position.

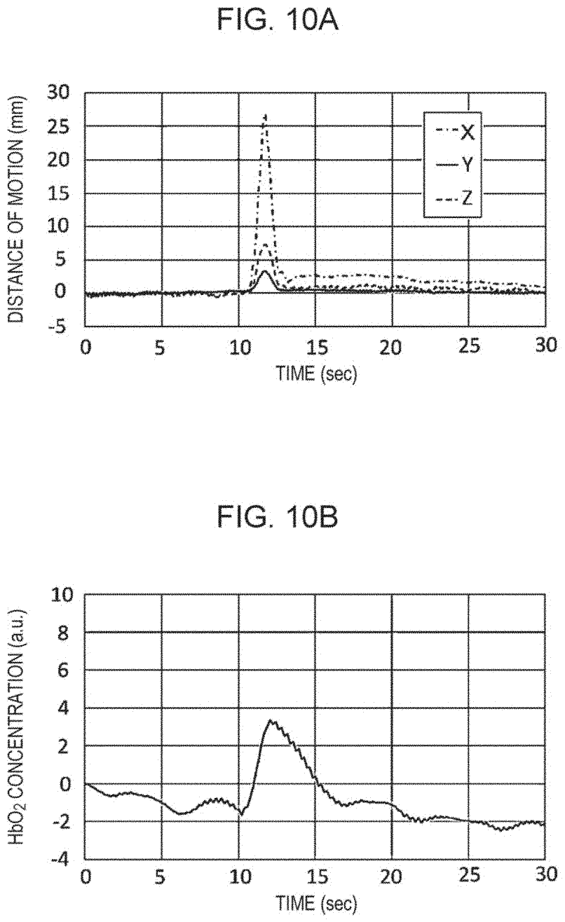

FIG. 10A illustrates a relationship between time and the distance of motion of the head. FIG. 10B illustrates a relationship between time and the oxygenated hemoglobin concentration. Referring to FIG. 10A, X denotes a change in the lateral direction, Y denotes a change in the vertical direction, and Z denotes a change in the depth direction. The distance of motion is a displacement from the initial value in each of the X, Y, and Z directions. Referring to FIG. 10B, the oxygenated hemoglobin concentration is an amount of change from the initial value thereof. The amount of change is represented in any unit.

Referring to FIGS. 10A and 10B, a larger change occurs in the oxygenated hemoglobin concentration in synchronization with about 11 to 12 seconds at which the head moved. It is thus noted that the body motion responsive to the motion of the head is correlated with the irregular change in the brain activity data.

It is also noted from the results in FIGS. 10A and 10B that a change in the oxygenated hemoglobin concentration occurs with a slight delay after the start of the motion of the head. It is also noted that the variation in the oxygenated hemoglobin concentration settles down with a delay after the end of the motion of the head. The following reasons are contemplated. (1) The brain is suspended within the cerebrospinal fluid in the skull. For this reason, the motion of the brain is delayed in time with respect to the movement of the skull. (2) During the measurement of the oxygenated hemoglobin concentration, time averaging is performed on a real-time basis. For this reason, the measurement is likely to be affected by an immediately preceding motion.

In accordance with results indicated in FIGS. 10A and 10B, the outputting of the brain activity data may be suspended after determining the validity if the following two conditions are satisfied. (1) The threshold is 5 mm in any of the X, Y, and Z directions. (2) The invalid time period is set to be from 0.5 before the concentration rises crossing the threshold to 4.5 seconds after the concentration falls crossing the threshold. Based on the two conditions, an irregular change in the measurement value of the oxygenated hemoglobin concentration may thus be removed.

The cerebral blood flow data was used as the biometric measurement data in the above example. The biometric measurement data is not limited to the cerebral blood flow data. The biometric measurement data may be at least one piece of data selected from the group of data on the scalp blood flow, pulse, perspiration, breathing, and temperature. The data may be acquired from the surface reflection component I1.

The present disclosure includes a program and method executed by the signal processing unit 122 and the signal processing unit 702.

Each biometric measurement apparatus of the present disclosure may be used in a camera or a measurement apparatus, acquiring internal information on the user in a non-contact fashion. For example, the biometric measurement apparatus may be applied for biosensing and medical sensing, sensing for a driver of an automobile, user sensing in a game machine and an attraction device, learner sensing in an educational institution, and worker sensing in a work place.

What is claimed is:

1. A biometric measurement apparatus comprising:
a light source that emits light onto a head portion of a user;
an image sensor;
a controller that controls the light source and the image sensor; and
a signal processor, wherein
the controller
causes the light source to emit the light and
causes the image sensor to output an image signal by causing the image sensor to detect at least part of reflected light returning from the head portion in response to emission of the light, and
the signal processor
generates brain activity data indicating a state of a brain of the user based on the image signal and
stops outputting the brain activity data based on at least one selected from the group consisting of the image signal and a sensor signal output from a sensor that detects a change in an environment surrounding the user, the change affecting the brain activity data.

2. The biometric measurement apparatus according to claim 1, wherein
the light is a light pulse, and
the controller causes the image sensor to output as the image signal a first signal that is obtained by detecting a component included in a reflected light pulse during a time period from start to end of a decrease of an intensity of the reflected light pulse, the reflected light pulse returning from the head portion in response to emission of the light pulse.

3. The biometric measurement apparatus according to claim 1, wherein
the controller causes the light source to repeatedly emit the light during a specific time period and causes the image sensor to repeatedly output the image signal during the specific time period, and
the signal processor
calculates a first value based on at least one selected from the group consisting of the image signal and the sensor signal and
stops outputting the brain activity data during a first time period of the specific time period, the first value satisfying a preset condition during the first time period.

4. The biometric measurement apparatus according to claim 3, wherein the signal processor outputs, during the first time period, a signal indicating that the brain activity data remains invalid.

5. The biometric measurement apparatus according to claim 3, wherein the signal processor outputs, during the first time period, data identical to the brain activity data that is generated prior to the first time period.

6. The biometric measurement apparatus according to claim 3, wherein the signal processor outputs, as the brain activity data during the first time period, data that is obtained by interpolating between the brain activity data generated prior to the first time period and the brain activity data generated subsequent to the first time period.

7. The biometric measurement apparatus according to claim 3, where the signal processor further stops outputting the brain activity data during at least a time period selected from the group consisting of a second time period prior to a start of the first time period and a third time period subsequent to an end of the second time period.

8. The biometric measurement apparatus according to claim 7, wherein the third time period is longer than the second time period.

9. The biometric measurement apparatus according to claim 3, wherein a frequency of calculating the first value is higher than or equal to a frequency of generating the brain activity data.

10. The biometric measurement apparatus according to claim 1, wherein the light is a light pulse,
the controller causes the image sensor to output as the image signal a second signal that is obtained by detecting a component included in a reflected light pulse prior to a start of a decrease of an intensity of the reflected light pulse, the reflected light pulse returning from the head portion in response to emission of the light pulse, and
the signal processor stops outputting the brain activity data based on the second signal.

11. The biometric measurement apparatus according to claim 10, wherein the signal processor
calculates, based on the second signal, a displacement from a reference position of the head portion or a motion speed of the head portion and
stops outputting the brain activity data when an absolute value of the displacement or an absolute value of the motion speed exceeds a threshold.

12. The biometric measurement apparatus according to claim 10, wherein the signal processor
calculates, based on the second signal, a luminance of the head portion or a change rate in the luminance of the head portion and
stops outputting the brain activity data when an absolute value of the luminance or an absolute value of the change rate in the luminance exceeds a threshold.

13. The biometric measurement apparatus according to claim 10, wherein the signal processor
calculates an area of a specific region in the head portion based on the second signal and
stops outputting the brain activity data if the area is smaller than a threshold.

14. The biometric measurement apparatus according to claim 1, wherein the signal processor
calculates a second value by using the brain activity data and
stops outputting the brain activity data when an absolute value of a change rate in the second value exceeds a threshold.

15. The biometric measurement apparatus according to claim 1, wherein the sensor is an acceleration sensor provided in the environment surrounding the user.

16. The biometric measurement apparatus according to claim 1, wherein the sensor is an illuminance sensor provided in the environment surrounding the user.

17. The biometric measurement apparatus according to claim 1, wherein the sensor includes at least one selected from the group consisting of a steering angle sensor, a gear position sensor, and a speed sensor, installed in a vehicle to be driven by the user.

18. A biometric measurement apparatus comprising:
a light source that emits light onto a head portion of a user;
an image sensor;
a controller that controls the light source and the image sensor; and
a signal processor, wherein
the controller
causes the light source to emit the light and
causes the image sensor to output an image signal by causing the image sensor to detect at least part of reflected light returning from the head portion in response to emission of the light, and the signal processor
generates brain activity data indicating a state of a brain of the user based on the image signal,
calculates reliability of the brain activity data based on at least one selected from the group consisting of the image signal and a sensor signal output from a sensor that detects a change in an environment surrounding the user, the change affecting the brain activity data, and
outputs reliability data indicating the reliability.

19. The biometric measurement apparatus according to claim 18, wherein the signal processor outputs the reliability data together with the brain activity data.

20. The biometric measurement apparatus according to claim 18, wherein the signal processor
calculates a first value based on at least one selected from the group consisting of the image signal and the sensor signal and
calculates the reliability that is lower as the first value is farther from a preset value.

21. The biometric measurement apparatus according to claim 18, wherein the signal processor
calculates a first value based on at least one selected from the group consisting of the image signal and the sensor signal and
calculates the reliability that is lower as a time period throughout which the first value exceeds a preset value is longer.

22. The biometric measurement apparatus according to claim 18, wherein the signal processor
calculates a first value based on at least one selected from the group consisting of the image signal and the sensor signal and
calculates the reliability that is lower as a time period throughout which the first value exceeds a preset value is longer within a constant time period.

23. The biometric measurement apparatus according to claim 18, wherein the sensor includes at least one selected from the group consisting of a steering angle sensor, a gear position sensor, and a speed sensor, installed in a vehicle to be driven by the user.

24. The biometric measurement apparatus according to claim 18, wherein
the light is a light pulse,
the controller causes the image sensor to output as the image signal a second signal that is obtained by detecting a component included in a reflected light pulse prior to a start of a decrease of an intensity of the reflected light pulse, the reflected light pulse returning from the head portion in response to emission of the light pulse, and
the signal processor calculates the reliability based on the second signal.

25. The biometric measurement apparatus according to claim 1, wherein
the controller causes the light source to repeatedly emit the light during a specific time period and causes the image sensor to repeatedly output the image signal during the specific time period, and
the signal processor
calculates a first value based on at least one selected from the group consisting of the image signal and the sensor signal and
stops outputting the brain activity data during a time period extending from an end of a delay time following a start of a first time period to an end of the first time period within the specific time period, the first value satisfying a preset condition during the first time period.

26. A biometric measurement method comprising:
causing a light source to emit light onto a head portion of a user;
causing an image sensor to output an image signal by causing the image sensor to detect at least part of reflected light returning from the head portion in response to emission of the light;
generating brain activity data indicating a state of a brain of the user based on the image signal; and
stopping outputting the brain activity data based on at least one selected from the group consisting of the image signal and a sensor signal output from a sensor that detects a change in an environment surrounding the user, the change affecting the brain activity data.

27. A biometric measurement method comprising:
causing a light source to emit light onto a head portion of a user;
causing an image sensor to output an image signal by causing the image sensor to detect at least part of reflected light returning from the head portion in response to emission of the light;
generating brain activity data indicating a state of a brain of the user based on the image signal;
calculating reliability of the brain activity data based on at least one selected from the group consisting of the image signal and a sensor signal output from a sensor that detects a change in an environment surrounding the user, the change affecting the brain activity data; and
outputting reliability data indicating the reliability.
